(19) United States
(12) Patent Application Publication
(10) Pub. No.: US 2026/0165777 A1
(43) Pub. Date: Jun. 18, 2026

(54) CATHETER INCLUDING A ROTATION MEMBER

(71) Applicant: Medtronic Ireland Manufacturing Unlimited Company, Dublin (IE)

(72) Inventors: Max H. Billard, Jacksonville, FL (US); Dishuan Chu, Rohnert Park, CA (US); Paul J. Coates, Corte Madera, CA (US)

(21) Appl. No.: 19/125,099

(22) PCT Filed: Oct. 9, 2023
(86) PCT No.: PCT/EP2023/077902

§ 371 Date: Apr. 28, 2025

Related U.S. Application Data
(60) Provisional application No. 63/381,428, filed on Oct. 28, 2022.

(51) Int. Cl. A61B 18/14; A61B 17/00; A61B 18/00

(52) U.S. Cl. CPC A61B 18/1492; A61B 2017/00867; A61B 2018/00214; A61B 2018/00404; A61B 2018/00577

(57) ABSTRACT

In some examples, a catheter includes an elongated body defining a longitudinal axis, the elongated body including an expandable portion, a therapy delivery element disposed on the expandable portion, wherein the expandable portion is configured to transform into an expanded configuration and place the therapy delivery element in apposition with a blood vessel wall at a first location, and a rotation member proximal to and separate from the expandable portion. The rotation member is configured to expand to cause the expandable portion to rotate about the longitudinal axis and place the therapy delivery element in apposition with the blood vessel wall at the second location.

This application claims the benefit of U.S. Provisional Patent Application Ser. No. 63/381,428, filed Oct. 28, 2022, the entire content of which is incorporated herein by reference.

### TECHNICAL FIELD

The present technology is related to neuromodulation therapy.

### BACKGROUND

Catheters have been proposed for use with various medical procedures. For example, a catheter can be configured to deliver neuromodulation therapy to a target tissue site to modify the activity of nerves at or near the target tissue site. The nerves can be, for example, sympathetic nerves. The sympathetic nervous system (SNS) is a primarily involuntary bodily control system typically associated with stress responses. Chronic over-activation of the SNS is a maladaptive response that can drive the progression of many disease states. For example, excessive activation of the renal SNS has been identified experimentally and in humans as a likely contributor to the complex pathophysiology of arrhythmias, hypertension, states of volume overload (e.g., heart failure), and progressive renal disease.

### SUMMARY

The present disclosure describes a catheter that includes one or more rotation members configured to position a therapy delivery element of the catheter at different rotational orientations, e.g., different locations around a perimeter of a blood vessel. Each rotation member is configured to expand to impart respective expanded configurations to a catheter body and reposition the therapy delivery element. Each rotation member is configured to rotate an expandable portion the catheter body about the longitudinal axis when the rotation member is expanded. In addition, in some examples, one or more rotation members are also configured to foreshorten an axis length the distal portion of the catheter body along the longitudinal axis when the rotation member is expanded. Thus, expanding one or more rotation members of the catheter may enable the distal portion of the catheter to position the therapy delivery element at different longitudinal and/or radial positions around an inner perimeter of a blood vessel.

The present disclosure also describes devices, systems, and methods for neuromodulation, such as renal neuromodulation.

The catheter including the one or more rotation members may provide improved control of the rotation of a distal portion of the catheter within the blood vessel and improved placement of one or more therapy delivery element at different longitudinal and/or radial positions within the blood vessel by using a torque imparted by transformation of the one or more rotation members to rotate the distal portion of the catheter. The devices, systems, and methods described in the disclosure may also reduce unintended effects on non-target tissue of the patient by improving the accuracy of the placements of the therapy delivery element and reducing the likelihood of the delivery of therapy to non-target tissue.

In some examples, the disclosure describes a catheter system comprising: a catheter comprising an elongated body defining a longitudinal axis, the elongated body comprising: an expandable portion; a therapy delivery element disposed on the expandable portion, wherein the expandable portion is configured to transform into an expanded configuration and place the therapy delivery element in apposition with a blood vessel wall at a first location; and a rotation member proximal to and separate from the expandable portion, the rotation member configured to expand to cause the expandable portion to rotate about the longitudinal axis and place the therapy delivery element in apposition with the blood vessel wall at a second location.

In some examples, the disclosure describes a catheter comprising: an elongated body defining a longitudinal axis, the elongated body comprising: an expandable portion; a therapy delivery element disposed on the expandable portion, wherein the expandable portion is configured to transform from a relatively low-profile configuration to a deployed configuration and place the therapy delivery element in apposition with a blood vessel wall at a first location; and a plurality of rotation members proximal to and separate from the expandable portion, each rotation member of the plurality of rotation members being configured to expand to cause the expandable portion to rotate about the longitudinal axis and place the therapy delivery element in apposition with the blood vessel wall at a corresponding location of a plurality of locations.

In some examples, the disclosure describes a method comprising: advancing a catheter through vasculature to a target tissue site within a blood vessel of a patient, the catheter comprising an elongated body defining a longitudinal axis, the elongated body comprising: an expandable portion; a therapy delivery element disposed on the expandable portion; and a rotation member proximal to and separate from the expandable portion; expanding the expandable portion to place the therapy delivery element in apposition to a vessel wall of the blood vessel at a first location; delivery, via the therapy delivery element, a therapy to tissue of the patient through the vessel wall at the first location; expanding the rotation member to rotate the expandable portion within the blood vessel and place the therapy delivery element in apposition to the vessel wall at a second location; and delivering, via the therapy delivery element, the therapy to tissue of the patient through the vessel wall at the second location.

Further disclosed herein is a catheter that includes an elongated body defining a longitudinal axis, the elongated body including an expandable portion, a therapy delivery element disposed on the expandable portion, wherein the expandable portion is configured to transform into an expanded configuration and place the therapy delivery element in apposition with a blood vessel wall at a first location, and a rotation member proximal to and separate from the expandable portion, wherein the rotation member is configured to expand to cause the expandable portion to rotate about the longitudinal axis and place the therapy delivery element in apposition with the blood vessel wall at the second location.

The details of one or more examples are set forth in the accompanying drawings and the description below. Other features, objects, and advantages will be apparent from the description and drawings, and from the claims.

### BRIEF DESCRIPTION OF THE DRAWINGS

Reference is made to the attached drawings, wherein elements have the same reference numeral designations represent similar elements throughout.

FIG. 1 is a partial schematic illustration of an example neuromodulation system.

FIG. 2A is a conceptual diagram illustrating the example distal portion of the elongated body of FIG. 1 positioned within a blood vessel in a collapsed configuration.

FIG. 2B is a conceptual diagram illustrating a partial cross-sectional view of the example distal portion of FIG. 2A taken along line A-A in FIG. 2A and along a plane parallel to the longitudinal axis of a catheter body.

FIG. 3A is a conceptual diagram illustrating the example distal portion of the elongated body of FIG. 1 positioned within a blood vessel in a first rotational configuration.

FIG. 3B is a conceptual diagram illustrating a partial cross-sectional view of the example distal portion of FIG. 3A taken along line B-B in FIG. 3A and along a plane parallel to the longitudinal axis of a catheter body.

FIG. 4A is a conceptual diagram illustrating the example distal portion of the elongated body of FIG. 1 positioned within a blood vessel in a second rotational configuration.

FIG. 4B is a conceptual diagram illustrating a partial cross-sectional view of the example distal portion of FIG. 4A taken along line D-D in FIG. 4A and along a plane parallel to the longitudinal axis of a catheter body.

FIG. 5A is a conceptual diagram illustrating a cross-sectional view of the example distal portion of FIG. 3A taken along line C-C in FIG. 3A and along a plane orthogonal to the longitudinal axis of the catheter body.

FIG. 5B is a conceptual diagram illustrating a cross-sectional view of the example distal portion of FIG. 4A taken along line E-E in FIG. 4A and along a plane orthogonal to the longitudinal axis of the catheter body.

FIG. 6 is a flow diagram illustrating an example method of repositioning a distal portion of a catheter within a blood vessel.

FIG. 7 is a flow diagram illustrating an example method of manufacturing an example rotation member of a catheter.

FIG. 8 illustrates an example technique for accessing a renal artery and modulating renal nerves with the system of FIG. 1 in accordance with some examples of the present disclosure.

FIG. 9 is a conceptual illustration of an example sympathetic nervous system (SNS) illustrating how the brain communicated with the body via the SNS.

FIG. 10 is an enlarged anatomic view of nerves innervating a left kidney to form the renal plexus surrounding the left renal artery.

FIG. 11 is an anatomic view of a human body depicting neural efferent and afferent communication between the brain and kidneys.

FIG. 12 is a conceptual view of a human body depicting neural efferent and afferent communication between the brain and kidneys.

FIG. 13 is an anatomic view of the arterial vasculature of a human.

FIG. 14 is an anatomic view of the venous vasculature of a human.

### DETAILED DESCRIPTION

The present disclosure describes catheters, as well as systems including such catheters and methods of using such catheters, that can be used for any suitable medical procedure, including neuromodulation, such as renal neuromodulation. Although neuromodulation and renal denervation is primarily described herein, devices, systems, and techniques described herein may be applied to other types of therapy, including other types of neuromodulation, such as neuromodulation performed on nerves other than the renal nerves, at sites other than within a renal vessel, or both. For instance, the techniques and devices described herein may be used to perform neuromodulation of nerves adjacent to at least one of (1) the renal arteries and/or their branches; (2) the celiac trunk and/or its branches (including the common hepatic artery and/or its branches, the left gastric artery and/or its branches, and the splenic artery and/or its branches); (3) the superior mesenteric artery and/or its branches; (4) the inferior mesenteric artery and/or its branches; or combinations of any two or more of these arteries and/or branches. In general, the devices, systems, and techniques described herein may be used to perform neuromodulation from within any suitable anatomical lumen that has nerves adjacent to the anatomical lumen. In addition, the systems, devices, and methods described herein may be useful for neuromodulation within a body lumen other than a vessel, for extravascular neuromodulation and/or for use in therapies other than neuromodulation.

As used herein, the terms “distal” and “proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction towards the clinician or clinician's control device.

Neuromodulation, such as renal denervation, may be accomplished using one or more of a variety of treatment modalities, including delivery of radiofrequency (RF) energy, microwave energy, ultrasound energy, thermal energy (e.g., direct thermal energy), optical energy, cryogenic cooling, a chemical agent, or the like. To perform intravascular neuromodulation, a neuromodulation catheter may be delivered to a blood vessel, such as a renal artery, of a patient. In some examples, a portion (e.g., a distal portion) of a catheter body of the neuromodulation catheter includes one or more therapy delivery elements (e.g., electrodes, ultrasound transducers, needles, fluid injection ports, or the like). While a distal portion of the catheter body is primarily referred to herein, in other examples, other portions of the catheter body can include the one or more therapy delivery element.

The distal portion of the catheter body may be configured to deploy from a relatively low profile delivery (or collapsed) configuration to a deployed configuration (e.g., a helical, spiral, basket, loop, or stent-like shape, an inflated state of a balloon, or the like). In the deployed configuration, the distal portion may be configured to position the one or more therapy delivery elements in apposition to a vessel wall to facilitate delivery of therapy to tissue of the patient, e.g., surrounding the vessel wall, perivascular tissue, or the like. The deployed configuration can also referred to herein as an expanded configuration because at least some parts of the catheter can expand radially away from a central longitudinal axis of a more proximal portion of the catheter, e.g., to bring the one or more therapy delivery elements into contact with a vessel wall or other tissue of interest.

An example catheter configured to be used for neuromodulation may include one or more therapy delivery elements (e.g., electrodes, ultrasound transducers, needles, fluid delivery ports, or the like) disposed on a portion of the example catheter. When a clinician places the distal portion at a target tissue site within a patient, the clinician may deliver therapy to tissue of the patient via the therapy delivery elements, e.g., for denervation procedures or the like. The clinician may deliver the therapy at different locations around a circumference of the blood vessel to increase the efficacy of the therapy. While a distal portion is primarily referred to herein, the therapy delivery elements can be on any suitable part of the catheter, and the rotation members described herein can be configured to rotate therapy delivery elements on any suitable part of a catheter.

The one or more therapy delivery elements are configured to deliver therapy to tissue of the patient, e.g., through the vessel wall of the blood vessel (e.g., to adventitial tissue or to perivascular tissue) at select locations around an inner perimeter of the blood vessel. In some examples, using a neuromodulation catheter, a clinician may deliver neuromodulation therapy (e.g., electrical or thermal energy or a chemical) to tissue adjacent to the blood vessel via therapy delivery elements position at multiple locations around the inner perimeter of the blood vessel. The clinician may deliver the neuromodulation therapy to targeted tissue adjacent to the blood vessel by delivering the therapy at the multiple locations along the inner perimeter of the blood vessel. The multiple locations may be separated by a predetermined angle (e.g., 90 degrees apart, 180 degrees apart). The clinician may delivery therapy to the multiple locations to, e.g., increase efficacy of the therapy, reduce a likelihood of occurrence of an unintended effect, and/or the like. While the therapy (e.g., energy or a chemical) is applied to the perimeter of the blood vessel, the target of the therapy or therapeutic effect can occur outside the blood vessel.

In some examples, the catheter may not be configured to deliver therapy to the different locations at the vessel wall from a single position and/or orientation within the blood vessel. For example, when expanded, the distal portion may place a therapy delivery element of the catheter at a first location of the multiple locations but not at a second location of the multiple locations. The clinician may need to re-position (e.g., advance, retract, and/or rotate) the neuromodulation catheter to deliver the therapy to all of the desired locations. In such examples, the clinician may rotate the catheter within the blood vessel to place the therapy delivery elements at the different locations around the perimeter (e.g., circumference) of the blood vessel. The material characteristics and the placement of the catheter through relatively tortuous vasculature may make it difficult for the clinician to transfer rotational force from a proximal portion of the catheter to the distal portion of the catheter or to control the rotation of the distal portion within the blood vessel.

With some neuromodulation catheters, the clinician may rotate a handle and/or a proximal portion of the catheter body to rotate the distal portion of the catheter body within the blood vessel. The clinician may apply a torque to the handle and/or the proximal portion of the catheter body by rotating the handle and/or the proximal portion about a longitudinal axis of the handle and/or the proximal portion of the catheter body. The catheter body may propagate the applied torque along a length of catheter body to the distal portion of the catheter body to rotate the distal portion and the therapy delivery element within the blood vessel (e.g., about a longitudinal axis of the distal portion of the catheter body). The handle may be of a different material than the catheter body and the transmission of torque across different materials with different material properties may lead to under-rotation of the distal portion of the catheter body and/or a reduction in the ability to precisely control the rotation of the distal portion of the catheter body. In addition, the catheter body is relatively flexible to allow for navigation of the catheter body through vasculature of the patient. The flexibility of the catheter body may cause the catheter body to resist transmission of the torque along the catheter body and lead to under-rotation of the distal portion of the catheter body and/or reduction in the ability to precisely control the rotation of the distal portion of the catheter body. In such examples, the clinician may need to over-rotate the handle and/or the proximal portion of the catheter body to rotate the distal portion of the catheter body within the blood vessel by a desired amount. For example, the clinician may need to rotate the handle and/or the proximal portion of the catheter body by more than 180 degrees to rotate the distal portion of the catheter body by 180 degrees. In some examples, rotation of the handle and/or the proximal portion of the catheter body by the clinician may cause the release of stress within the catheter and cause over-rotation of the distal portion of the catheter body or “whipping,” e.g., a delayed response by the catheter to rotational movement by the clinician, which can be caused by spring torsion.

The under-rotation or over-rotation of the distal portion of the catheter body and/or the reduction in the ability to precisely control the rotation of the distal portion of the catheter body may lead to one or more difficulties with the neuromodulation procedure. For example, the clinician may need to spend a relatively long period of time to position the distal portion of the catheter body to a desired location due to the increase difficulty in precisely manipulating the distal portion of the catheter body. While the neuromodulation may still be effective, there may be a reduction in the efficacy of the neuromodulation therapy if the therapy is not delivered to an intended location (e.g., a location in the blood vessel proximate the target nerves for ablation) and/or delivered therapy to an unintended location (e.g., non-target nerves or other non-target tissue). The delivery of neuromodulation therapy to the non-target tissue may lead to unintended outcomes.

In examples described herein, a catheter includes a catheter body, a therapy delivery element disposed on an expandable portion of the catheter body, and a rotation member configured to rotate the expandable portion of the catheter body. The rotation of the expandable portion can cause the therapy delivery element to rotate from a first rotational position to a second, different rotational position. The rotation member may be separate from the expandable portion. For example, the rotation member may be proximal to the expandable portion and/or the distal portion of the catheter body.

The catheter body may include one or more rotation members, each rotation member being configured to rotate the therapy delivery element to different rotational positions. Expansion and/or collapse of different rotation members may cause the expandable portion of the catheter body to rotate between different rotational orientations and cause the therapy delivery element to be positioned at different rotational locations within the blood vessel. The clinician may expand and/or contract the rotation members by retracting or advancing a guide member (e.g., a guidewire, an inner catheter, an outer sheath, or the like) along the catheter body and/or by advancing the catheter body relative to the guide member. When expanded each rotation member may define a helix, loop, lasso, circle, spiral, biased curvature, waveform, or another curvilinear or non-curvilinear shape.

The rotation member may be configured to cause the expandable portion and, in some cases, the distal portion of the catheter body to rotate to cause the therapy delivery element to be positioned at different locations within the blood vessel by applying a torque to the distal portion of the catheter body in response to the expansion and/or collapse of the one or more rotation members. The clinician may expand each rotation member by retracting the guide member along the catheter body (or otherwise causing relative movement of the guide member and the catheter body) to a position proximal to the respective rotation member. In some examples, as the rotation member expands from a non-expanded (e.g., compressed) configuration to a respective expanded configuration, the rotation member may foreshorten an axis length of the distal portion of the catheter body along the longitudinal axis. The foreshortening is a result of the radially outward expansion of distal portion of the catheter body caused by the rotation member.

While some example neuromodulation catheters described herein may include one rotation member, other example neuromodulation catheters described herein may include two or more rotation members. Each of the different rotation members may be disposed along a length of the catheter and along a longitudinal axis of the catheter. One or more rotation members (e.g., a first rotation member) of the different rotation members may be relatively distal to another rotation member (e.g., a second rotation member).

The example devices, methods, and systems described herein provide several benefits over other neuromodulation catheters. Expanding the member to reposition the therapy delivery elements within the blood vessel may allow for precise control of the placement of therapy delivery elements at the multiple locations, e.g., by removing resistance to the rotation from the material characteristics or the tortuosity of the catheter body. The precise control of the placement of therapy delivery elements may lead to increased efficacy of the neuromodulation therapy and decreased likelihood of the occurrence of unintended effects as a result of the neuromodulation therapy.

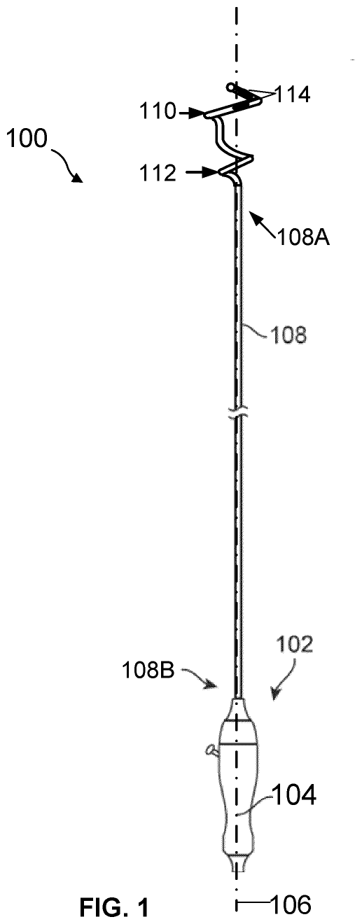

FIG. 1 is a partially schematic illustration of an example neuromodulation catheter system 100 (“system 100”). System 100 includes a neuromodulation catheter 102, which includes a handle 104, a control device (not shown in FIG. 1), and an elongated body 108 also referred to as “catheter body 108” or “elongated member 108”) attached to handle 104. Elongated body 108 includes a distal portion 108A and a proximal portion 108B. Distal portion 108A includes an expandable portion 110 and a rotation member 112 disposed proximal to expandable portion 110. In some examples, expandable portion 110 is separated from rotation member 112 by a relatively-straight portion of distal portion 108A. The relatively-straight portion of distal portion 108A may maintain a relatively-straight or collapsed configuration when one or more of expandable portion 110 or rotation member 112 is expanded to a respective expanded configuration. In other examples, expandable portion 110 and rotation member 112 are directly adjacent to each other. Expandable portion 110 is configured to rotate about longitudinal axis 106 in response to an expansion of rotation member 112.

In the example shown in FIG. 1, catheter 102 includes one or more therapy delivery elements 114 disposed along expandable portion 110. Each of therapy delivery elements 114 is configured to deliver therapy to tissue of a patient, e.g., to neuromodulate target nerve(s) of the patient. Therapy delivery elements 114 may include, but are not limited to, one or more electrodes, one or more ultrasound transducers, one or more needles, one or more heat or cryo-therapy delivery devices (e.g., balloons) or one or more injection ports configured to deliver a therapeutic agent. Therapy delivery elements 114 may be connected to a therapy source (e.g., an electrical signal generator, a source of a therapeutic agent, a cryogenic therapeutics source, or the like) via electrical conductor(s) and/or lumen(s) defined by elongated body 108 and/or handle 104. Although FIG. 1 illustrates catheter 102 as having two therapy delivery elements 114, other example catheters may include a single therapy delivery element 114 or three or more therapy delivery elements 114, such as four electrodes.

Elongated body 108 may have any suitable outer diameter, and the diameter can be constant along the length of elongated body 108 or may vary along the length of elongated body 108. In some examples, elongated body 108 can be 2, 3, 4, 5, 6, or 7 French or another suitable size.

Distal portion 108A of elongated body 108 is configured to be advanced within an anatomical lumen of a human patient to locate therapy delivery elements 114 at a target tissue site within or otherwise proximate to the anatomical lumen. For example, elongated body 108 may be configured to position distal portion 108A within a blood vessel, a ureter, a duct, an airway, or another naturally occurring lumen within the human body. The examples described herein focus on the anatomical lumen being a blood vessel, such as a renal vessel, but it will be understood that similar techniques may be used with other anatomical lumens. In certain examples, intravascular delivery of distal portion 108A includes percutaneously inserting a guidewire (not shown in FIG. 1) into a vessel of a patient and moving elongated body 108 and/or expandable portion 110 along the guidewire until expandable portion 110 reaches a target tissue site (e.g., a renal artery). For example, distal portion 108A of elongated body 108 may define a passageway for engaging the guidewire for delivery of expandable portion 110 using over-the-wire (OTW) or rapid exchange (RX) techniques. In other examples, neuromodulation catheter 102 can be a steerable or non-steerable device configured for use without a guidewire. In still other examples, neuromodulation catheter 102 can be configured for delivery via a guide member (e.g., a guide catheter, an outer sheath (not shown in FIG. 1), or other guide device).

In the example illustrated in FIG. 1, catheter 102 is configured to assume a relatively low-profile delivery configuration (also referred to as a collapsed configuration or non-expanded configuration) in which distal portion 108A defines a relatively smaller radial extent (a relatively low profile, such as a relatively linear configuration), and a radially expanded configuration in which expandable portion 110 of distal portion 108A defines a relatively larger radial extent. Distal portion 108A may be delivered through vasculature of the patient to the target tissue site in the delivery configuration. Expandable portion 110 is configured to transform between the relatively low profile (e.g., collapsed) delivery configuration and a deployed configuration, which is also referred to herein as a radially-expanded configuration or an expanded configuration. Expandable portion 110 may be configured to self-expand within a blood vessel of a patient, e.g., via a shape-memory element (e.g., a shape memory tube) of elongated body 108. Expandable portion 110 may be restrained in the delivery configuration by a guide member. The clinician may retract the guide member proximally relative to expandable portion 110 to un-restrain expandable portion 110 and cause expandable portion 110 transform from the delivery configuration to the expanded configuration.

In some examples, in the radially expanded configuration, expandable portion 110 defines a helical, a spiral, a loop, a basket, or a stent-like configuration. In the radially expanded configuration, expandable portion 110 is configured to position one or more therapy delivery elements of the plurality of therapy delivery elements 114 near a vessel wall, e.g., in apposition with the vessel wall.

In some examples, expandable portion 110 may be expanded or may self-expand as a result of proximal retraction of a guide member from distal portion 108A. The clinician may retract the guide member to a location along distal portion 108A proximal to expandable portion 110 and distal to rotation member 112 to cause expandable portion 110 to expand and to keep rotation member 112 in the collapsed configuration. In the expanded configuration, expandable portion 110 may place therapy delivery elements 114 at a first set of locations relative to the vessel wall, e.g., corresponding to a first rotational location.

Once at the target tissue site, expandable portion 110 can be expanded to place therapy delivery elements 114 in apposition with the vessel wall at the target tissue site. System 100 may then deliver, provide, or facilitate neuromodulation therapy at the target tissue site, e.g., through the vessel wall at the target tissue site to target tissue adjacent to the blood vessel. The neuromodulation therapy may include, but is not limited to, radiofrequency (RF) energy, microwave energy, ultrasound energy, a therapeutic agent (e.g., a chemical ablation agent), cryogenic energy, or the like.

Rotation member 112 is positioned proximal to expandable portion 110. As discussed in further detail below, rotation member 112 can be integrally formed part of catheter 102, e.g., embedded in a wall of elongated body 108, or can be separate from and positioned in catheter 102. In the example shown in FIG. 1, rotation member 112 located along distal portion 108A. In other examples, however, rotation member 112 can be disposed on other parts of catheter 102.

When expandable portion 110 is in the expanded configuration, the clinician may further retract the guide member to a position proximal to a rotation member 112 to cause the corresponding rotation member 112 to expand to an expanded configuration. Expansion of the rotation member 112 causes the corresponding rotation member 112 to apply a torque to expandable portion 110 and cause expandable portion 110 to rotate from the first rotational orientation to a second rotational orientation. Rotation of expandable portion 110 to the second rotational orientation may cause therapy delivery elements 114 to be rotate to different locations along an inner perimeter of the vessel wall.

In some examples, as illustrated in FIG. 1, distal portion 108A includes one rotation member 112. In other examples, distal portion 108A includes two or more rotation members 112 longitudinally spaced apart from each other and the clinician may expand each rotation member 112 of the two or more rotation member 112 to rotate expandable portion 110 to two or more corresponding rotational configurations within the blood vessel.

Expansion of each rotation member 112 may cause rotation of expandable portion 110 within the blood vessel and about longitudinal axis 106 to position therapy delivery elements 114 at different locations relative to the vessel wall (e.g., different circumferential positions) corresponding to the respective rotational orientation. When expanded, each rotation member 112 may impart a corresponding expanded configuration to distal portion 108A. Expansion of each rotation member 112 may apply a torque to portions of distal portion 108A and/or expandable portion 110 distal to the respective rotation member 112 relative to longitudinal axis 106 and cause distal portion 108A and/or expandable portion 110 to rotate within the blood vessel about longitudinal axis 106.

The application of torque from rotation member 112 to expandable portion 110 may cause a portion of distal portion 108A distal to rotation member 112 and/or expandable portion 110 to rotate about longitudinal axis 106, e.g., in a same direction as the torque. Rotation of distal portion 108A and/or expandable portion 110 may cause therapy delivery elements 114 disposed on distal portion 108A and/or expandable portion 110 to rotate about longitudinal axis 106, e.g., in the same direction as the torque. In some examples, expansion of each of one or more rotation members 112 can also cause a foreshortening of an axis length of distal portion 108A along the longitudinal axis 106.

Rotation member 112 may include a shape memory material (e.g., nitinol) that, when unrestrained, is configured to self-expand from a collapsed configuration to an expanded configuration. Elongated body 108 may, for example, include an elongated tube (e.g., a helical hollow strand) formed from a shape memory material and configured to radially expand away from longitudinal axis 106 and cause expandable portion 110 and/or one or more rotation members 112 to expand to the respective expanded configurations when unrestrained by a guide member.

In some examples, rotation member 112 may be restrained by the guide member (e.g., a guidewire disposed within a rotation member lumen defined by rotation member 112, a sheath disposed over distal portion 108A, a guidewire disposed in a catheter lumen, or the like). The guide member may be advanced distally along longitudinal axis 106 and into or over distal portion 108A to restrain expandable portion 110 and rotation member 112 in a collapsed delivery configuration. The guide member may be retracted/withdrawn proximally (or otherwise moved relative to elongated body 108) to un-restrain expandable portion 110 and/or rotation member 112 and cause expandable portion 110 and/or rotation member 112 to transform into an expanded configuration.

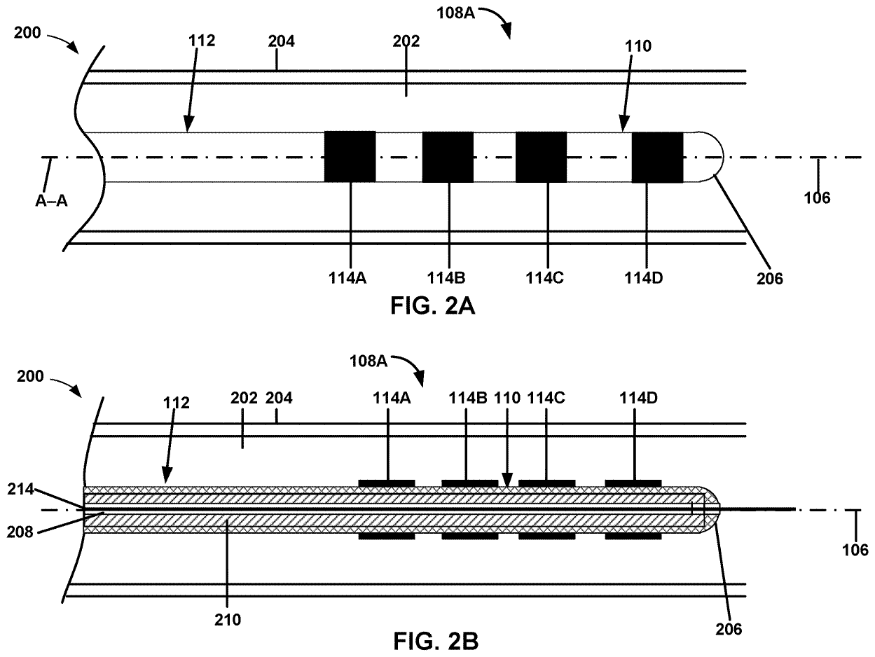

FIG. 2A is a conceptual diagram illustrating the example distal portion 108A of elongated body 108 of FIG. 1 positioned within a blood vessel 202 in a collapsed configuration 200. As illustrated in FIG. 2A, when distal portion 108A is in collapsed configuration 200 (also referred to as “delivery configuration 200”), expandable portion 110 and rotation member 112 assume relatively low profile configurations with relatively smaller radial extents. Therapy delivery elements 114A-114D (collectively referred to as “therapy delivery elements 114”) are disposed on expandable portion 110 and separated along longitudinal axis 106 in the example shown in FIG. 2A, but can have other configurations in other examples. For example, an example distal portion 108A may include one to three therapy delivery elements 114 or five or more therapy delivery elements 114. In some examples, therapy delivery elements 114 may include electrodes, ultrasound transducers, injection ports, or the like. A distal end of expandable portion 110 may define distal tip 206. Distal tip 206 may facilitate navigation of distal portion 108A within the vasculature of the patient to blood vessel 202. In some examples, distal tip 206 may be atraumatic, e.g., to avoid puncturing vessel wall 204 of blood vessel 202 during navigation of distal portion 108A within blood vessel 202.

FIG. 2B is a conceptual diagram illustrating a schematic cross-sectional view of the example distal portion 108A of FIG. 2A taken along line A-A in FIG. 2A and along a plane parallel to longitudinal axis 106 of elongated body 108.

In the example shown in FIG. 2B, elongated body 108 includes an elongated tube 210 (e.g., a tube of shape-memory material) including a plurality of portions, each portion being configured to, when expanded impart a corresponding expanded configuration to expandable portion 110 and/or one or more rotation members 112. As illustrated, expandable portion 110 may overlap with one or more therapy delivery elements 114. In other examples, as illustrated in FIG. 2B, rotation member 112 is positioned proximal to expandable portion 110 and/or therapy delivery elements 114. In each of these examples, expansion of one or more additional rotation members 112 causes additional rotation of expandable portion 110.

Each rotation member 112 may impart a corresponding expanded configuration to a portion of distal portion 108A proximal to expandable portion 110 and cause expandable portion 110 to rotate to a corresponding rotational configuration. In some examples, two or more, or all of rotation members 112 have the same or similar expanded configurations (e.g., a helical, a loop shape, a lasso shape, a circular shape, a spiral shape, or the like) and/or dimensions (e.g., pitch, outer diameter, number of coils/revolutions, direction of winding). In other examples, two or more of the expanded configurations of the corresponding expanded portions have different shapes (e.g., a helical shape, a loop shape, a lasso shape, a circular shape, a spiral shape, or the like), and/or dimensions as one or more other expanded configurations. Each expanded configuration of the one or more rotation members 112 may be configured to cause expandable portion 110 to place one or more therapy delivery elements of therapy delivery elements 114 at different rotational and/or longitudinal positions, e.g., of a corresponding rotational configuration.

Elongated tube 210 may be formed from a shape-memory material (e.g., nitinol) configured to transform into an expanded configuration within blood vessel 202. Elongated tube 210 may be heat set to a shape of one or more of an expanded configuration of expandable portion 110 or expanded configurations of rotation members 112. For example, elongated tube 210 may be heat set to define expandable portion 110 and rotation member 112 in expanded configurations, as illustrated in FIG. 1. In some examples, the shape-memory material may be formed into an elongated tube (e.g., a helical hollow strand (HHS®) tube available from Fort Wayne Metals Research Products, L.L.C., Fort Wayne, Indiana, a hypotube) and may define catheter lumen 208.

As illustrated in FIG. 2B, catheter lumen 208 can be configured to receive a guidewire 214. Guidewire 214 may facilitate navigation of elongated body 108 within blood vessel via an OTW techniques, a RX technique, or the like. In some examples, guidewire 214, when longitudinally overlapping one or more of expandable portion 110 or one or more rotation members 112, restrains the corresponding portions of distal portion 108A in the collapsed configuration. Guidewire 214 is configured to be advanced distally or retracted proximally along longitudinal axis 106 within catheter lumen 208. A clinician may advance or retract guidewire 214 to cause expandable portion 110 and/or at least a portion of rotation member 112 to transform to the collapsed configuration or to a corresponding expanded configuration. Each of expandable portion 110 and/or at least a portion of rotation member 112 may be expanded to the expanded configuration by expansion of a corresponding portion of elongated tube 210. For example, the clinician may retract guidewire 214 to a first position proximal to expandable portion 110 and distal to or otherwise aligned with rotation member 112 to allow expandable portion 110 to expand into an expanded configuration and place therapy delivery elements 114 in apposition with vessel wall 204. The clinician may further retract guidewire 214 to a second position proximal to at least one rotation member 112 to allow the at least one rotation member 112 to expand and rotate expandable portion 110 about longitudinal axis 106. The clinician may then further retract guidewire 214 proximally to un-restrain one or more other rotation members 112 and cause expandable portion 110 to further rotate about longitudinal axis 106. Expansion of each of the one or more other rotation members 112 may cause the respective rotation member 112 to apply a torque to expandable portion 110 and cause expandable portion 110 and therapy delivery elements 114 to rotate within blood vessel 202.

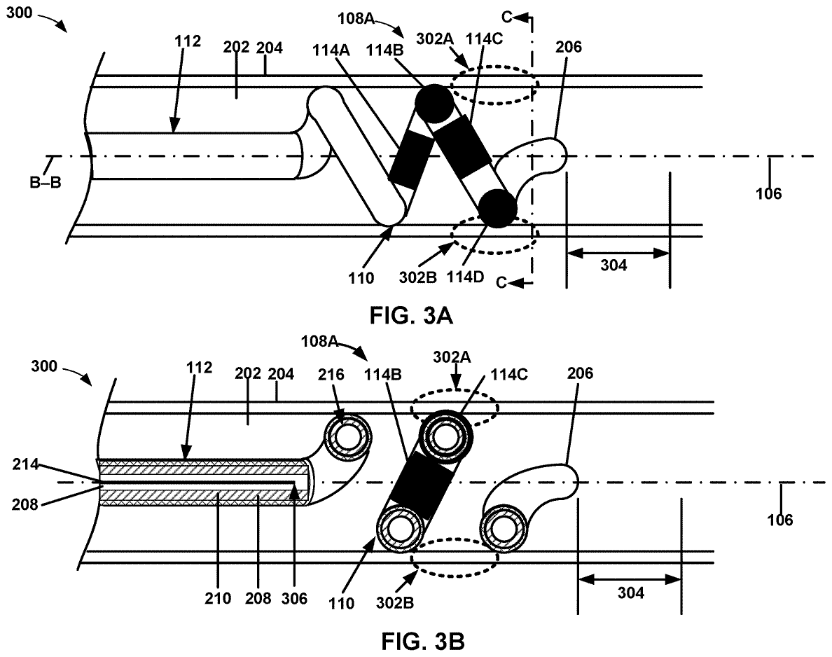

FIG. 3A is a conceptual diagram illustrating the example distal portion 108A of elongated body 108 of FIG. 1 positioned within blood vessel 202 in a first rotational configuration 300. As illustrated in FIG. 3A, expandable portion 110 is transformed into a first helical, loop, or spiral shape. While the example expandable portion 110 illustrated in FIG. 3A is configured to transform into a helical, loop, lasso, circular, or spiral shape, other example expandable portions may transform into a hoop shape, a basket shape, an expanded balloon, an expanded stent configuration, or other shapes configured to place therapy delivery elements 114 in apposition with vessel wall 204.

Locations 302A, 302B (also referred to as “locations 302”, “rotational positions 302”) indicate portions of vessel wall 204 around the perimeter (e.g., circumference) of blood vessel 202. As an example, a clinician may deliver therapy to target tissue adjacent to blood vessel 202 through vessel wall 204 at each of locations 302 to create an area of influence including the target tissue. In some examples, the area of influence may be around the entire outer circumference of blood vessel 202. In some examples, locations 302 have a same longitudinal position within blood vessel 202 relative to longitudinal axis 106, while in other examples, locations 302 have different longitudinal positions relative to longitudinal axis 106.

Locations 302 have different radial positions around a perimeter of blood vessel 202 and each of locations 302 may be separated by a predetermined angle (e.g., by 180 degrees, by 90 degrees, or the like) from another location 302. In some examples, locations 302 may be 180 degrees apart, which may improve efficacy of therapy by allowing for the delivery of therapy by therapy delivery elements 114 to create opposing ablation patterns around the inner perimeter of vessel wall 204. In some examples, the predetermined angle may be between about 45 degrees and about 180 degrees apart. For a particular neuromodulation procedure, there may be two locations 302 or three or more locations 302 around the inner circumference of blood vessel 202. Locations 302 may be evenly disposed around the inner circumference of vessel wall 204 or may be unevenly disposed, such that locations 302 are biased towards a particular portion of the inner perimeter (e.g., inner circumference) of vessel wall 204.

Rotation member 112 is configured to rotate expandable portion 110 to a plurality of rotational configurations. Each rotational configuration of the plurality of rotational configurations may be separated by a predetermined angle. In each rotational configuration, expandable portion 110 may be configured to place therapy delivery elements 114 in apposition with vessel wall 204 at a different selection of locations 302.

In some examples described herein, the predetermined angle is described with respect to the circumference of blood vessel 202. In some examples, the predetermined angle may be an angle of rotation of expandable portion 110 outside of the body of the patient (in the absence of external forces applied by a vessel wall or the like) when rotation member 112 transforms from the collapsed configuration to the expanded configuration. For example, the angles can be along an imaginary circle, a central longitudinal axis of elongated body 108 extending through the center of the imaginary circle. In some examples, the angle of rotation of expandable portion 110 outside of the body of the patient is about 45 degrees to about 180 degrees. The predetermined angle outside of the body of the patient may correspond to an amount of torque applied to expandable portion 110 by rotation member 112 as a result of an expansion of rotation member 112. In some examples, depending on the size (e.g., diameter) and/or location of blood vessel 202, expansion of rotation member 112 may cause a same amount of torque to rotate expandable portion 110 by different predetermined angles. for example, the predetermined angle of rotation for expandable portion 110 inside a blood vessel 202 may be greater than, less than, or equal to the predetermined angle of rotation for expandable portion 110 outside of the body of the patient.

The predetermined angle may represent an effective angle of rotation of expandable portion 110 about longitudinal axis 106. For example, an angle of rotation of 315 degrees in a counterclockwise direction around longitudinal axis 106 has a same effective angle of rotation of 45 degrees as an angle of rotation of 45 degrees in a clockwise direction around longitudinal axis 106. As another example, an angle of rotation of 405 degrees in a clockwise direction around longitudinal axis 106 has a same effective angle of rotation as an angle of rotation of 45 degrees in the clockwise direction around longitudinal axis 106. Expansion of rotation member 110 may cause expandable portion 110 to rotate clockwise or counterclockwise around longitudinal axis 106 until a new rotational configuration of expandable portion 110 is separated from a prior rotational configuration by the effective angle of rotation. In some examples, expansion of rotation member 110 may cause expandable portion 110 to over-rotate (i.e., rotate more than 360 degrees) about longitudinal axis 106 to place expandable portion 110 at a new rotational configuration separated from the prior rotational configuration by the effective angle of rotation.

When guidewire 214 is withdrawn from elongated body 108 to a position proximal expandable portion 110, elongated tube 210 (e.g., as shown in FIG. 2B) may expand and cause expandable portion 110 to transform into an expanded configuration. When expanded, expandable portion 110 may cause at least one of therapy delivery elements 114 (e.g., therapy delivery element 114D) to be placed adjacent to or in apposition with vessel wall 204 at one or more locations of locations 302 (e.g., at location 302B). Therapy delivery elements 114 are configured to deliver therapy to target tissue of the patient through vessel wall 204 at the one or more locations of locations 302. For example, as illustrated in FIG. 3A, therapy delivery element 114D can be used to deliver therapy through vessel wall 204 at location 302B.

Distal portion 108A may foreshorten along longitudinal axis 106 by a distance 304 relative to the delivery configuration as a result of expansion of expandable portion 110, e.g., shown in FIGS. 2A and 2B. That is, the expansion of expandable portion 110 can cause a reduction in an axis length of distal portion 108A along longitudinal axis 106. Distance 304 may be predetermined in some examples, and may depend on one or more dimensions of the expanded configuration of expandable portion 110 in first expanded configuration including but are not limited to, a pitch, a maximum outer diameter, or a number of coils/revolutions of expandable portion 110 in the expanded configuration, as well as a size (e.g., diameter) of vessel 202.

FIG. 3B is a conceptual diagram illustrating a schematic cross-sectional view of the example distal portion 108A of FIG. 3A taken along line B-B in FIG. 3A and along a plane parallel to longitudinal axis 106 of elongated body 108. As illustrated in FIG. 3B, guidewire 214 is retracted proximally along longitudinal axis 106 to a first position 306. First position 306 is proximal to expandable portion 110 and distal to rotation member 112. Retracting guidewire 214 to first position 306 un-restrains a portion of elongated tube 210 radially overlapping with expandable portion 110 and causes expandable portion 110 to deploy into a radially expanded configuration. That is, expandable portion 110 radially expands away from longitudinal axis 106 into an expanded configuration.

The process for expanding expandable portion 110 can also be reversed to collapse expandable portion 110. For example, guidewire 214 can be configured to be advanced distally from first position 306 towards distal tip 206. Advancing guidewire 214 towards distal tip 206 causes expandable portion 110 to transform from the expanded configuration to a collapsed configuration (e.g., as illustrated in FIGS. 2A and 2B). Transformation of expandable portion 110 into the collapsed configuration causes catheter 102 to transform from first rotational configuration 300 to collapsed configuration 200.

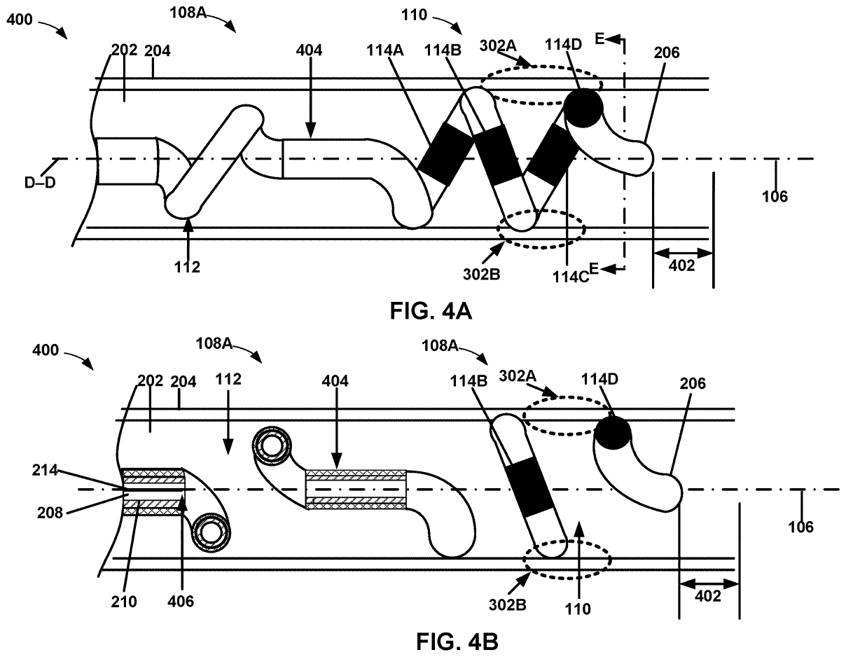

FIG. 4A is a conceptual diagram illustrating example distal portion 108A of catheter body 108 of FIG. 1 positioned within blood vessel 202 in a second rotational configuration 400. When unrestrained by proximal retraction of guidewire 214, rotation member 112 radially expands away from longitudinal axis 106 and causes expandable portion 110 to rotate from first rotational configuration 300 to second rotational configuration 400. The expanded configuration of rotation member 112 may be a helical, loop, lasso, circular, or spiral shape, as illustrated in FIG. 4A, a biased curvature, a waveform shape, or another curvilinear or non-curvilinear shape configured to impart a torque to expandable portion 110. Expandable portion 110 and rotation member 112 are separated by a relatively linear or otherwise non-expanded portion 404 in the example shown in FIG. 4A.

Expansion of rotation member 112 to an expanded configuration causes expandable portion 110 to rotate, retract, and/or advance within blood vessel 202 relative to, e.g., first rotational configuration 300. Expansion of rotation member 112 applies torque to expandable portion 110 and causes expandable portion 110 to rotate within blood vessel 202. Rotation of expandable portion 110 places therapy delivery elements 114 (e.g., therapy delivery element 114D) in apposition to vessel wall 204 at different locations 302 (e.g., at location 302A) within blood vessel 202. The different locations 302 may correspond to second rotational orientation 400 and may be different relative to the locations 302 shown in FIGS. 3A and 3B when distal portion 108A was in the expanded configuration 300. For example, the different locations 302 can be longitudinally and/or circumferentially offset from the first locations.

In some examples, as illustrated in FIG. 4A, when expanded, rotation member 112 may define a helix, a loop, a lasso, a circle, a spiral, or the like revolving around longitudinal axis 106. In some examples, distal portion 108A may include two or more rotation members 112, each rotation member 112 defining an expanded configuration defining helixes, loops or spirals wound in a same or different directions.

Expansion of rotation member 112 may foreshorten an axis length of distal portion 108A along longitudinal axis 106 by a distance 402. In some examples, distance 402 is predetermined and is based on a pitch, an outer diameter, a number of coils/revolutions, and/or any other dimensions of second expanded configuration 112B, a dimension of blood vessel 202, and/or a predetermined amount of torque applied to expandable portion 110. In some examples, distance 402 is less than or equal to a length of one of distances 304 along longitudinal axis 106. In some examples, distance 402 may be less than, greater than, or equal to distance 304.

FIG. 4B is a conceptual diagram illustrating a schematic cross-sectional view of the example distal portion 108A of FIG. 4A taken along line D-D in FIG. 4A and along a plane parallel to longitudinal axis 106 of elongated body 108. Guidewire 214 is configured to be proximally retracted along longitudinal axis 106 to second position 406 proximal to rotation member 112. Proximal retraction of guidewire 214 to second position 406 un-restrains at least one rotation member 112 and allows the unrestrained rotation member 112 to radially expand away from longitudinal axis 106. While FIG. 4B illustrates and describes expansion of rotation member 112 primarily with reference to a single rotation member 112, other example distal portion 108A may include two or more rotation members 112. Each of the two or more rotation members 112 may be expanded in a similar manner as described below to rotate expandable portion 110 into different corresponding rotational configurations. The rotation members can be longitudinally spaced along longitudinal axis 106, such that as a guide member is withdrawn proximal to the respective rotation member, the rotation member expands to cause expandable member 110 to rotate.

The expanded configuration assumed by rotation member 112 may have a same or different pitch, number of coils/revolutions, outer diameter, or other dimensions as the expanded configuration assumed by expandable portion 110. In some examples, the expanded rotation member 112 may have a tighter pitch, a fewer number of coils/revolutions, and/or a smaller outer diameter than the expanded expandable portion 110. The dimensions of expanded rotation member 112 may affect an amount (e.g., an angle) of rotation of expandable portion 110 and/or therapy delivery elements 114. In some examples, the dimensions of expanded rotation member 112 affects an amount of foreshortening of the axis length of distal portion 108A along longitudinal axis 106, e.g., by distance 402.

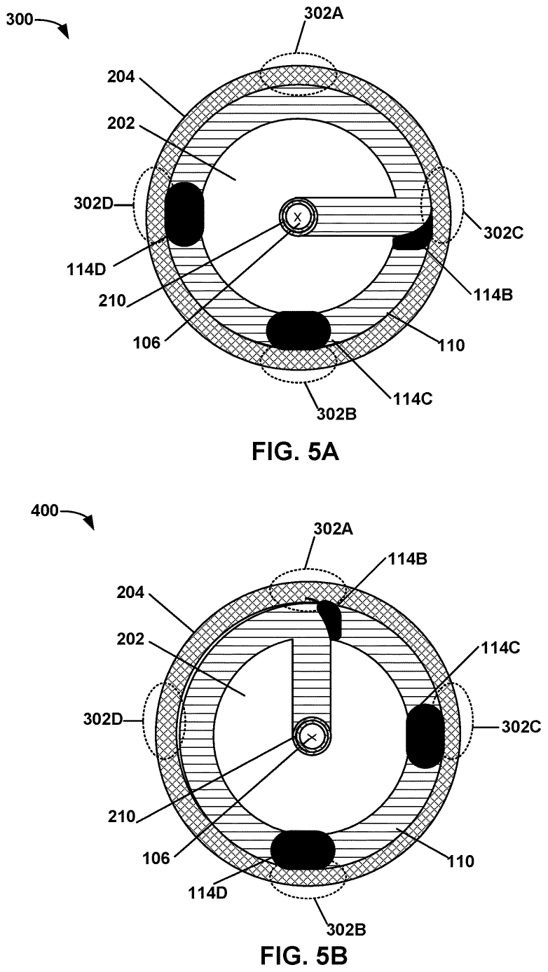

FIG. 5A is a conceptual diagram illustrating a schematic cross-sectional view of distal portion 108A of FIG. 3A in first rotational configuration 300 taken along line C-C in FIG. 3A and along a plane orthogonal to longitudinal axis 106 of catheter 102. FIG. 5B is a conceptual diagram illustrating a schematic cross-sectional view of distal portion 108A of FIG. 4A in second rotational configuration 400 taken along line E-E in FIG. 4A and along a plane orthogonal to longitudinal axis 106 of the distal portion 108A.

FIGS. 5A and 5B illustrates four locations 302A-302D (collectively referred to as “locations 302”) on vessel wall 204 and around the perimeter of blood vessel 202. Locations 302 may be selected to facilitate delivery of therapy around a desired portion of the perimeter of blood vessel 202. Collectively, the areas of influence of each of locations 302 may be combined to allow the delivery of therapy to the tissue of the patient around the entire outer perimeter of blood vessel 202 at the target tissue site. The delivery of the therapy around the entire outer circumference of blood vessel 202 may improve efficacy of the therapy and/or reduce the likelihood and/or severity of any unintended results, e.g., by reducing an amount of therapy delivered to the target tissue site to affect the tissue around the entire outer circumference of blood vessel 202 relative to the delivery of the therapy to relatively fewer locations 302. Blood vessel 202 may include two locations 302, three locations 302, or four or more locations 302 disposed around the circumference of blood vessel 202.

As illustrated in FIGS. 5A and 5B, as rotation member 112 become un-restrained (e.g., by retraction of guidewire 214, an outer sheath, or other guide members along longitudinal axis 106, to second position 406, or to another position along elongated body 108), rotation member 112 may expand and cause expandable portion 110 to rotate about longitudinal axis 106. Expansion of rotation member 112 causes an application of torque expandable portion 110 to rotate expandable portion 110 from first rotational configuration 300 to second rotational configuration 400. In each of the different rotational configurations (e.g., first rotational configuration 300, second rotational configuration 400), expandable portion 110 may place at least one of therapy delivery elements 114 in apposition with vessel wall 204 of blood vessel 202 at one or more of locations 302.

In some examples, elongated body 108 includes a locking member proximal to at least one of rotation members 112. The locking member may include, but is not limited, a locking stylet, a locking guidewire, a locking sheath, an expandable member (e.g., an expandable balloon), or the like. The locking member may restrain a portion of elongated body 108 proximal to the at least one rotation member 112, e.g., to direct the torque caused by the expansion of the at least one rotation member 112 to act distally towards expandable portion 110 and cause expandable portion 110 to rotate about longitudinal axis 106. In some examples, the locking member may restrain the proximal portion of elongated body 108 to prevent transmission of the torque to the proximal portion of elongated body 108 and/or rotation of the proximal portion of elongated body 108 due to the expansion of the at least one rotation member 112.

In some examples, as illustrated in FIGS. 5A and 5B, in one of the rotational configurations (e.g., first rotational configuration 300), therapy delivery elements 114 may be disposed at a first set of predetermined locations 302 around the circumference of blood vessel 202. When torque is applied by rotation member 112 (e.g., due to expansion of rotation member 112), expandable portion 110 may be rotated to another of the rotational configurations (e.g., second rotational configuration 400) and place at least one therapy delivery element 114 (e.g., therapy delivery element 114C) in apposition with vessel wall 204 at a second set of locations 302 different from the first set. For example, the second set can include a location 302 (e.g., location 302C, location 302D) in a same rotational position as a location 302 previously occupied by another therapy delivery element 114 (e.g., therapy delivery element 114B, therapy delivery element 114D) but at a different longitudinal position. In another example, the second set can include a location 302 in a same longitudinal position as a location 302 previously occupied by another therapy delivery element 114 but at a different rotational position.

In some examples, as illustrated in FIGS. 2A-5B and described above, elongated tube 210 may be a part of elongated body 108 and/or permanently disposed within elongated body 108. In some examples, elongated tube 210 may be disposed within a catheter lumen defined by elongated body 108 and may be advanced and/or retracted relative to elongated body 108 and within the catheter lumen. In such examples, the clinician may select an elongated tube 210 from a plurality of different elongated tubes 210. Each elongated tubes 210 may have a different number of expanded configurations and/or expanded configurations having different dimensions and the clinician may select an elongated tube 210 to be used within system 100 based on the dimensions of the target tissue area, the dimensions of catheter 102, an intended area of effect at the target tissue area, the desired dimensions of expanded expandable portion 110, the desired dimensions of expanded rotation member 112, or the like.

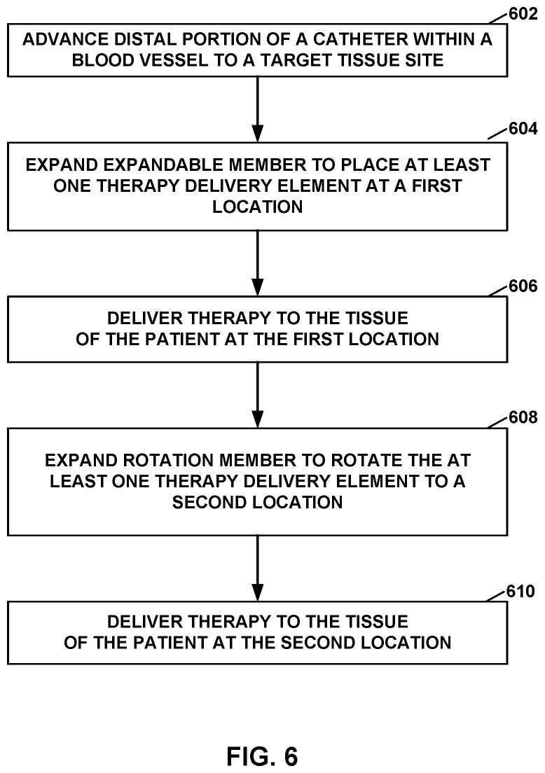

FIG. 6 is a flow diagram illustrating an example method of repositioning an example distal portion 108A of a catheter 102 within a blood vessel 202 using a rotation member (e.g., rotation member 120) described herein. While the example method illustrated in FIG. 6 is primarily described herein with reference to an example expandable portion 110 configured to expand into a helical, loop, or a spiral shape as illustrated in FIGS. 2A-5B, the example method of FIG. 6 may be applied to example expandable portions configured to expand into other shapes (e.g., a loop shape, a basket shape, an expanded balloon, an expanded stent configuration, any other shape configured to place therapy delivery elements 114 in apposition with vessel wall 204).

A clinician may advance distal portion 108A of catheter 102 within blood vessel 202 to a target tissue site within blood vessel 202 (602). The clinician may insert a guide member (e.g., guidewire 214, a guide sheath) into the vasculature of the patient via an incision on the patient and navigate the guide member to the target tissue site. Once the clinician determines the guide member is at the target tissue site, the clinician may advance catheter 102 along the guide member to the target tissue site. The clinician may navigate the guide member and/or catheter 102 within the vasculature using one or more imaging techniques (e.g., fluoroscopy, X-ray imaging, or the like). Elongated body 108 and/or rotation member 112 of catheter 102 define catheter lumen 208 configured to retain the guide member (e.g., guidewire 214). In other examples, the guide member (e.g., a sheath) may be configured to retain elongated body 108. The guide member may be configured to restrain rotation members 112 in a collapsed configuration as catheter 102 is navigated to the target tissue site.

The clinician may expand expandable portion 110 to place therapy delivery elements 114 at a first location (604). The clinician may cause expandable portion 110 of catheter 102 to assume an expanded configuration and place at least one therapy delivery element 114 in apposition with vessel wall 204 at a first location of locations 302.

The clinician may retract the guide member proximally to a first position 306 to un-restrain expandable portion 110. First position 306 may be proximal to expandable portion 110 and distal to rotation member 112. When unrestrained, the shape memory of a first portion of elongated tube 210 within elongated body 108 may cause expandable portion 110 to transform into the expanded configuration. When expanded, expandable portion 110 may place one or more of therapy delivery elements 114 in apposition with vessel wall 204 of blood vessel 202 at the first predetermined set of locations 302. Expansion of expandable portion 110 may cause a foreshortening of an axis length of distal portion 108A along longitudinal axis 106 by distance 304.

The clinician may deliver therapy to tissue of the patient at the first location (606). Each of therapy delivery elements 114 of catheter 102 is configured to deliver the therapy to the patient, e.g., to target nerve(s) and/or tissue of the patient through vessel wall 204 at locations 302. Once the clinician determines that the at least one therapy delivery element 114 is disposed at the first set of location 302, the clinician may delivery the therapy to the target nerve(s) and/or tissue through vessel wall 204 at the first set of locations 302. The therapy may include, but is not limited to, RF energy, ultrasound energy, electrical stimulation signals, cryogenic energy, chemicals, or the like. Therapy delivery elements 114 may deliver the therapy to the tissue of the patient via transmission of electrical stimulation signals via an electrode, via a chemical and/or fluid injection port, or the like.

The clinician may expand rotation member 112 to rotate the at least one therapy delivery element 114 to a second location (608). The clinician may expand rotation member 112 to rotate expandable portion 110 and place the at least one therapy delivery element 114 is in apposition with vessel wall 204 at the second location of locations 302.

The second location of locations 302 may be at a different radial position around the inner circumference of blood vessel 202 than the first location of locations 302. The first and second rotational positions may be separated by a predetermined angle (e.g., by 90 degrees, by 180 degrees, or the like). The first location and the second location of locations 302 may be separated by 180 degrees to increase efficacy of the therapy. In some examples, the second location may be proximal to the first location relative to longitudinal axis 106 (e.g., due to foreshortening of distal portion 108A and/or expandable portion 110 during the expansion of rotation member 112). In some examples, first rotational configuration 300 and second rotational configuration 400 may be selected such that when therapy is delivered to vessel wall 204 in when distal portion 108A is in the rotational positions, the combined area of influence of the therapy may encompass tissue around the entire outer circumference of blood vessel 202.

The clinician may expand rotation member 112 by at least retracting the guide member proximally to second position 406 to un-restrain rotation member 210. Rotation member 112 may expand and apply a torque to expandable portion 110. The applied torque may cause expandable portion 110 to rotate within blood vessel 202 and rotate the at least one therapy delivery element 114 from the first location of locations 302 to the second location of locations 302. The expansion of rotation member 112 may foreshorten the axis length of distal portion 108A along longitudinal axis 106 by distance 402.

The clinician may deliver therapy to the tissue of the patient at the second location (610). The clinician may deliver therapy via the at least one therapy delivery element 114 to the second location of location 302. In some examples, as described above, catheter 102 may include two or more rotation members 112 and may be configured to deliver therapy at two or more rotational positions, e.g., by further proximal retraction of the guide member to un-restrain additional rotation members 112.

In some examples, after the clinician delivers therapy to the tissue of the patient when catheter 102 is at the second rotational position, the clinician may transform expandable portion 110 into the collapsed delivery configuration, e.g., by advance the guide member distally along elongated body 108 and expandable portion 110. The clinician may then navigate catheter 102 to a second target tissue site within the patient and perform the example method describes in steps 602-610 at the second target tissue site.

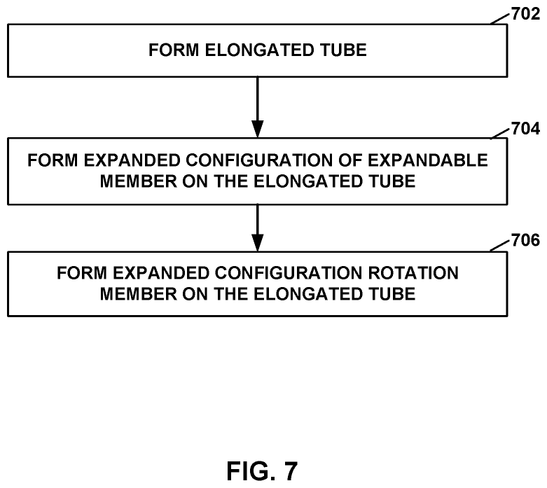

FIG. 7 is a flow diagram illustrating an example method of manufacturing an example rotation member 112 of a catheter 102. While the example method of FIG. 7 illustrates the manufacturing of a single rotation member 112, the steps of the example method may be used to manufacture two or more rotation members 112 of an example catheter 102.

A manufacturer may form an elongated tube 210 (702). The manufacturer may form a shape-memory material (e.g., nitinol) into elongated tube 210. Elongated tube 210 may include an HHS tube, a hypotube, or the like. In some examples, such as in an HHS tube, the manufacturer may wrap a plurality of shape-memory wires (e.g., around a mandrel) to form elongated tube 210.

The manufacturer may form an expanded configuration of expandable portion 110 (also referred to as “first expanded configuration”) on elongated tube 210 (704). The manufacturer may form the first expanded configuration on elongated tube 210 by shaping a portion of elongated tube 210 into the expanded configuration, e.g., under the application of heat to allow elongated tube 210 to retain a shape memory of the expanded configuration and to cause expandable portion 110 to transform from a collapsed configuration into the expanded configuration in response to a threshold temperature. The threshold temperature may be less than or equal to an internal body temperature of a patient or an average internal body temperature of patients. When expanded, the first expanded configuration is configured to cause expandable portion 110 to assume a radially expanded configuration and place at least one therapy delivery element 114 in apposition with vessel wall 204.

The manufacturer may determine dimensions of first expanded configuration based at least in part on desired dimensions of distal portion 108A and/or expandable portion 110 in the radially expanded configuration at one or more target tissue sites (e.g., an outer diameter, a pitch, and/or a number of coils/revolutions of expandable portion 110 in the radially expanded configuration at one or more target tissue sites), the dimensions of blood vessel 202 at the one or more target tissue sites (e.g., an inner circumference of blood vessel 202, a diameter of blood vessel 202), a positions of one or more of locations 302 at the target tissue site, and/or a threshold foreshortening distance value for distance 304. In some examples, the relationship between the dimensions of expandable portion 110 in the first expanded configuration and foreshortening distance 304 may be illustrated in Equation 1:

$$π * X * C + Y * C = Z . \qquad ( Equation 1 )$$

In Equation 1, Z represents a length of expandable portion 110 in a collapsed delivery configuration along longitudinal axis 106, C represents a number of coils/revolutions defined by expandable portion 110 in the first expanded configuration, Y represents a pitch of expandable portion 110 in the first expanded configuration, and X represents an outer diameter of expandable portion 110 in the first expanded configuration. Foreshortening distance 304 is represented by the term “x*X*C.” The manufacturer may determine distance 304 and/or dimensions of expandable portion 110 in first expanded configuration based at least in part on Equation 1.

The manufacturer may form an expanded configuration of rotation member 112 on elongated tube 210 (706). The manufacturer may form the expanded configuration of rotation member 112 at a position on elongated tube 210 proximal to the expanded configuration of expandable portion 110.

The manufacturer may determine the dimensions of the expanded configuration of rotation member 112 based at least in part on a desired angle of rotation of at least one of therapy delivery elements 114 (e.g., greater than or equal of 45 degrees, less than or equal to 180 degrees), a threshold foreshortening value of distance 402, dimensions of blood vessel 202 at the target tissue site, a desired level of torque to be applied to expandable portion 110, and/or a threshold level of torque to be applied to expandable portion 110. In some examples, rotation member 112 may be configured to rotate the at least one of therapy delivery elements 114 by 90 degrees and distance 402 may be less than or equal to a length of therapy delivery element 114. The manufacturer may determine distance 402 and/or dimensions of expanded configuration of rotation member 112 based at least in part on Equation 1 as applied to rotation member 112 in the expanded configuration.

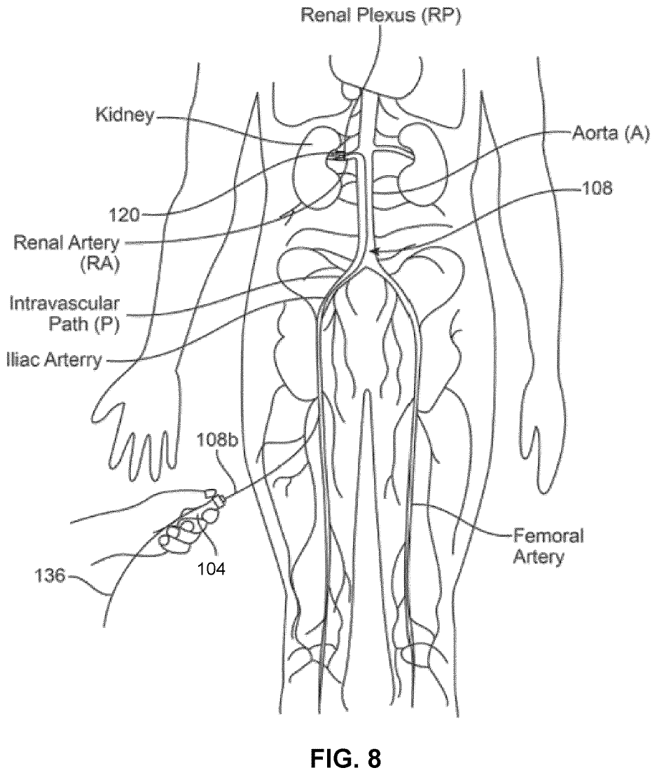

FIG. 8 illustrates an example technique for accessing a renal artery and modulating renal nerves with the system of FIG. 1 in accordance with some examples of the present disclosure. While FIG. 8 illustrates the use of catheter 102 for renal neuromodulation, catheter 102 may be used for other therapies and treatments within another blood vessel or other hollow anatomical body within the human body. Catheter 102 is configured to delivery energy (e.g., RF energy, ultrasound energy, electrical stimulation energy, or the like) to one or more target tissue sites within a renal vessel. Catheter 102 provides access to the renal plexus (RP) through an intravascular path (P), such as a percutaneous access site in the femoral (illustrated), brachial, radial, or axillary artery to the target tissue sites within a respective renal artery (RA). By manipulating proximal portion 108B or elongated body 108 from outside the intravascular path (P), a clinician may advance at least distal portion 108A of elongated body 108 through the sometimes-tortuous intravascular path (P) and remotely manipulate distal portion 108A (FIG. 1) of elongated body 108. Distal portion 108A may be remotely manipulated by the clinician using the handle 104.

In the example illustrated in FIG. 8, distal portion 108A is delivered intravascularly to the treatment site using an inner member 136 in an over-the-wire (OTW) technique. Inner member 136 may be internal to catheter 102 (e.g., a guide wire, inner catheter, or the like) or external to catheter 102 (e.g., an outer sheath or the like). In some examples, inner member 136 is a navigation wire. Catheter 102 may define a passageway for receiving inner member 136 for delivery of catheter 102 using either an OTW or an RX technique. At the treatment site, inner member 136 can be at least partially withdrawn or removed relative to catheter 102 and distal portion 108A can transform into an expanded configuration (e.g., a helical configuration, a spiral configuration, or the like) for delivering neuromodulation therapy. In other examples, elongated body 108 may be self-steerable such that expandable portion 110 may be delivered to the target tissue site without the aid of inner member 136.

Renal modulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for a period of months, years, or decades) or short-term (e.g., for periods of minutes, hours, days, or weeks). Renal neuromodulation is expected to contribute to the systemic reduction of sympathetic tone or drive and/or benefit at least some specific organs and/or other bodily structures innervated by sympathetic nerves. Accordingly, renal neuromodulation is expected to be useful in treating clinical conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end state renal disease, inappropriate fluid retention in heart failure, cardio-renal syndrome, polycystic kidney disease, polycystic ovary syndrome, osteoporosis, erectile dysfunction, and sudden cardiac death, among other conditions.

Renal neuromodulation can be electrically induced or induced in another suitable manner through the delivery of energy (RF energy, ultrasound energy, microwave energy, or the like). The target tissue site can be within or otherwise proximate to a renal lumen (e.g., a renal artery, a ureter, a renal pelvis, a major renal calyx, a minor renal calyx, or another suitable structure), and the target tissue site can include tissue at least proximate to a wall o f the renal lumen. For example, with regard to a renal artery, a treatment procedure can include modulating nerves in the renal plexus, which lay intimately within or adjacent to the adventitia of the renal artery. The following discussion provides further details regarding patient anatomy and physiology as it may relate to renal denervation therapy. This section is intended to supplement and expand upon the previous discussion regarding the relevant anatomy and physiology, and to provide additional context regarding the disclosed technology and the therapeutic benefits associated with renal denervation. For example, several properties of the renal vasculature may inform the design of the target tissue devices and associated methods for achieving renal neuromodulation via intravascular access and impose specific design requirements for such devices. Specific design requirements may include accessing the renal artery, positioning distal portion 108A within the renal artery, delivering the therapy to targeted tissue, and/or effectively modulating the renal nerves with the therapy delivery device.

As noted previously, the sympathetic nervous system (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operates through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons are therefore called presynaptic (or preganglionic) neurons, while peripheral sympathetic neurons are called postsynaptic (or postganglionic neurons).

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up- and down-regulating many homeostatic mechanisms in living organisms. Fibers from the SNS innervate tissues in almost every organ system, providing at least some regulatory function to physiological features as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

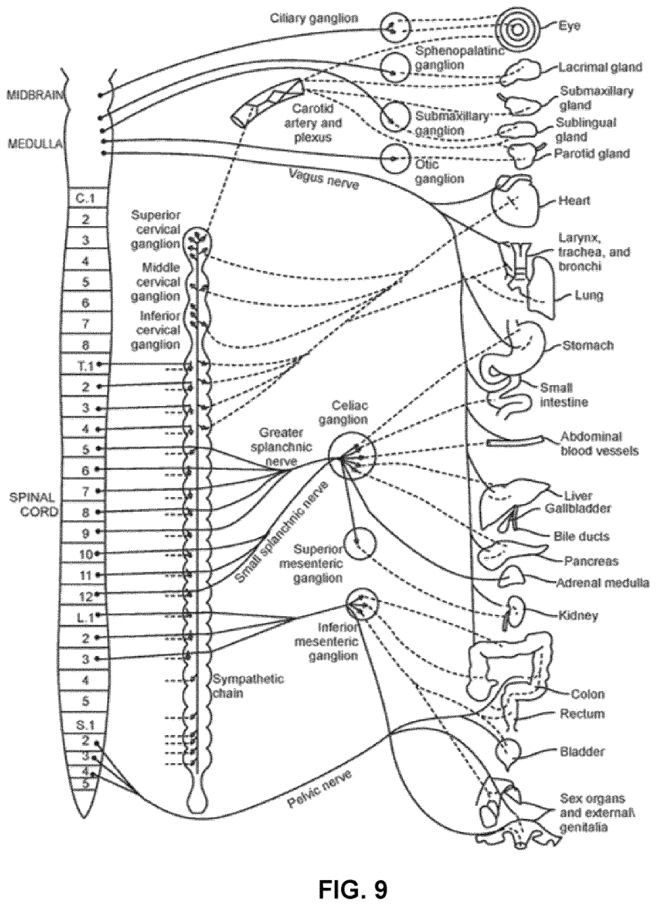

FIG. 9 is a conceptual illustration of an example sympathetic nervous system (SNS) illustrating how the brain communicated with the body via the SNS. As shown in FIG. 9, the SNS provides a network of nerves that allows the brain to communicate with the body. Sympathetic nerves originate inside the vertebral column, e.g., toward the middle of the spinal cord in the intermediolateral cell column (or lateral horn), beginning at the first thoracic segment of the spinal cord and are thought to extend to the second or third lumbar segments. Because SNS cells begin in the thoracic and lumbar regions of the spinal cord, the SNS is said to have a thoracolumbar outflow. Axons of sympathetic nerves leave the spinal cord through the anterior rootlet/root. The axons pass near the spinal (sensory) ganglion, where the axons enter the anterior rami of the spinal nerves. However, unlike somatic innervation, the axons separate out through white rami connectors which connect to either the paravertebral (which lie near the vertebral column) or prevertebral (which lie near the aortic bifurcation) ganglia extending alongside the spinal column.

To reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other component of the peripheral nervous system, these synapses are made at sites called ganglia, discussed above. The cell that sends its fiber to the ganglion is called a preganglionic cell, while the cell whose fiber leaves the ganglion is called a postganglionic cell. As mentioned previously, the preganglionic cell of the SNS is located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle, and inferior), which send sympathetic nerve fibers to the head and thorax organs, and the celiac and mesenteric ganglia, which send sympathetic fibers to the gut.

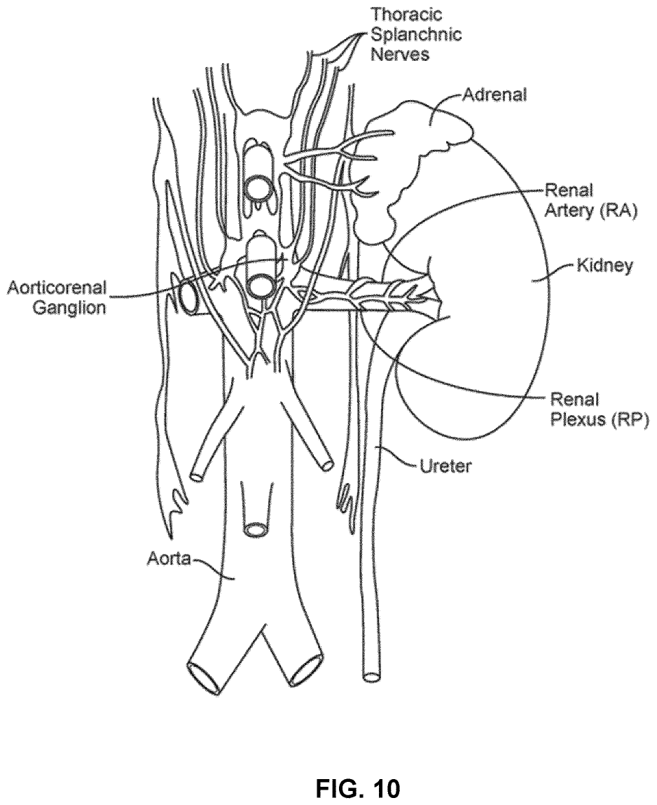

FIG. 10 is an enlarged anatomic view of nerves innervating a left kidney to form the renal plexus surrounding the left renal artery. As FIG. 10 shows, the kidney is innervated by the renal plexus (RP), which is intimately associated with the renal artery. The renal plexus (RP) is an autonomic plexus that surrounds the renal artery and is embedded within the adventitia of the renal artery. The renal plexus (RP) extends along the renal artery and is embedded within the adventitia of the renal artery. Fibers contributing to the renal plexus (RP) arise from the celiac ganglion, the superior mesenteric ganglion, the aorticorenal ganglion and the aortic plexus. The renal plexus (RP), also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia to become the lesser splanchnic nerve, the least splanchnic nerve, the first lumbar splanchnic nerve, the second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus (RP) and are distributed to the renal vasculature.

Messages travel through the SNS in a bi-directional flow. Efferent messages may trigger changes in different parts of the body simultaneously. For example, the sympathetic nervous system may accelerate heart rate, widen bronchial passages, decrease motility (movement) of the large intestine, constrict blood vessels, increase peristalsis in the esophagus, cause pupil dilation, piloerection (goose bumps) and perspiration (sweating), or raise blood pressure. Afferent messages carry signals from various organs and sensory receptors in the body to other organs and, particularly, the brain.

Hypertension, heart failure, and chronic kidney disease are a few of the many disease states that result from chronic activation of the SNS, especially the renal sympathetic nervous system. Chronic activation of the SNS is a maladaptive response that drives the progression of theses disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure) and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration late, and left ventricular ejection fraction. These findings support the notion that treatment regimens that are designed to reduce renal sympathetic stimulation have the potential to improve survival in patients with heart failure.

Both chronic and end state renal disease in some patients are characterized by heightened sympathetic nervous activation. In patients with end state renal disease, plasma levels of norepinephrine above the media have been demonstrated to be predictive for both all-cause death and death from cardiovascular disease. This can also be true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well-known adverse consequences of chronic sympathetic over activity, such as hypertension, left ventricular hypertrophy, ventricular arrhythmias, sudden cardiac death, insulin resistance, diabetes, and metabolic syndrome.

Sympathetic nerves to the kidneys terminate in the blood vessels, the juxtaglomerular apparatus, and the renal tubules. Stimulation of the renal sympathetic nerves cause increased renin release, increased sodium (Na⁺) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation may be a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). However, the current pharmacologic strategies can have significant limitations including limited efficacy, compliance issues, side effects, and others.

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication.

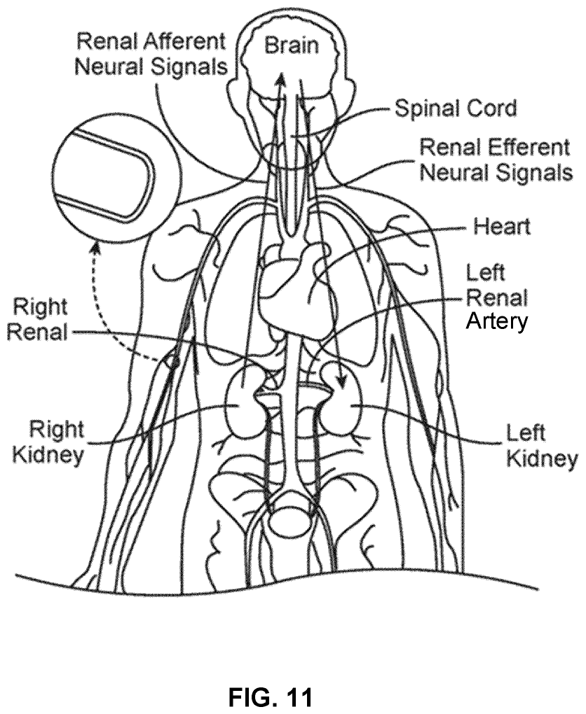

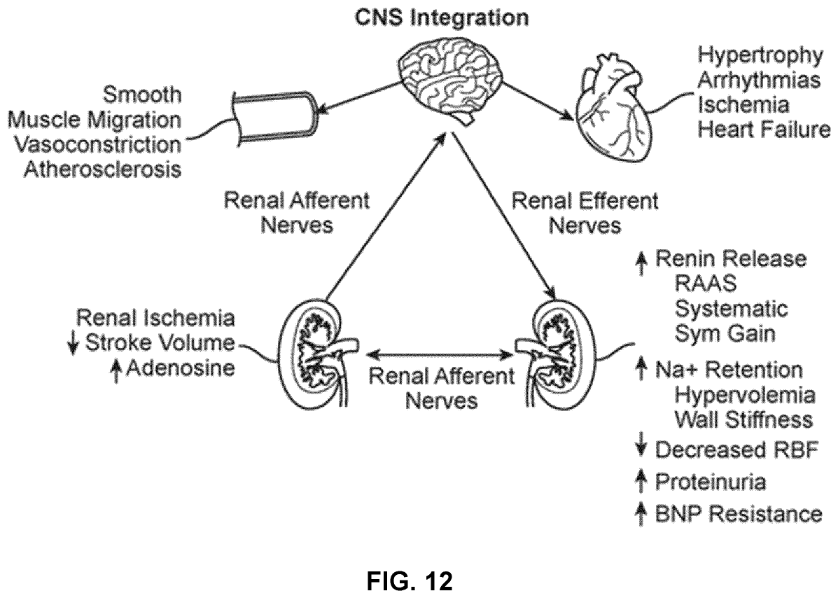

FIG. 11 is an anatomic view of a human body depicting neural efferent and afferent communication between the brain and kidneys. FIG. 12 is a conceptual view of a human body depicting neural efferent and afferent communication between the brain and kidneys. As shown in FIGS. 11 and 12, the afferent communication might be from kidney to the brain or might be from one kidney to the other kidney (via the central nervous system). These afferent signals are centrally integrated and may result in increased sympathetic outflow. This sympathetic drive is directed towards the kidneys, thereby activating the RAAS and inducing increased renin secretion, sodium retention, volume retention, and vasoconstriction. Central sympathetic over activity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end state renal disease, inappropriate fluid retention in heart failure, cardio-renal syndrome and sudden cardiac death. Since the reduction of afferent neural signals contributing to the systemic reduction of sympathetic tone/drive, renal denervation might also be useful in treating other conditions associate with systemic sympathetic hyperactivity. Accordingly, renal denervation may also benefit other organs and bodily structures innervated by sympathetic nerves, including those identified in FIG. 11. For example, as previously discussed, a reduction in central sympathetic drive may reduce the insulin resistance that afflicts people with metabolic syndrome and Type II diabetics. Additionally, patients with osteoporosis may also be sympathetically activated and might also benefit from the down regulation of sympathetic drive that accompanies renal denervation.

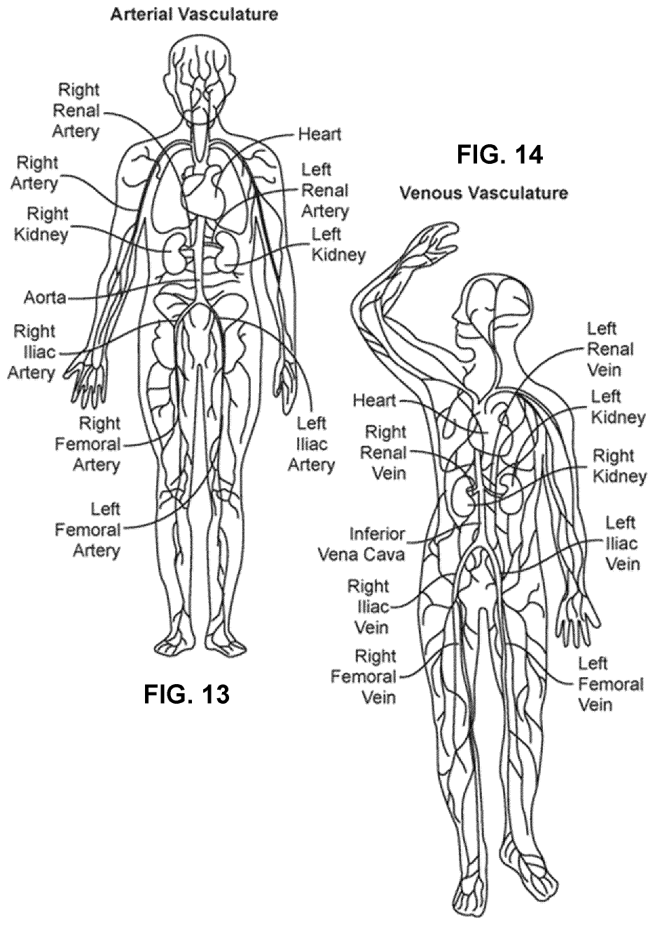

In accordance with the present technology neuromodulation of a left and/or right renal plexus (RP), which is intimately associated with a left and/or right renal artery, may be achieved through intravascular access. FIG. 13 is an anatomic view of the arterial vasculature of a human. As FIG. 13 shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

FIG. 14 is an anatomic view of the venous vasculature of a human. As FIG. 14 shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

The femoral artery may be accessed and cannulated at the base of the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters (e.g., catheter 102) introduced via these access points may be passed through the subclavian artery on the left side (or via the subclavian and brachiocephalic arteries on the right side), through the aortic arch, down the descending aorta and into the renal arteries using standard angiographic techniques. Other access sites can also be used to access the arterial system.

Since neuromodulation of a left and/or right renal plexus (RP) may be achieved in accordance with the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, and the like. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Further, some patients include multiple left renal arteries and/or right renal arteries. Apparatus, systems, and methods for achieving renal neuromodulation via intravascular access should account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. For example, navigation can be impeded by the tight space within a renal artery, as well as tortuosity of the artery. Furthermore, establishing consistent contact is complicated by patient movement, respiration, and/or the cardiac cycle because these factors may cause significant movement of the renal artery relative to the aorta, and the cardiac cycle may transiently distend the renal artery (i.e., cause the wall of the artery to pulse).

The neuromodulatory apparatus may also be configured to allow for adjustable positioning and repositioning of distal portion 108A and expandable portion 110 (FIG. 1) within the renal artery since location of treatment may also impact clinical efficacy. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging.

As noted above, an apparatus positioned within a renal artery should be configured so that expandable distal portion 108A of catheter 102 may intimately contact the vessel wall and/or extend at least partially through the vessel wall. Renal artery vessel diameter, DRA, typically is in a range of about 2-10 mm, with most of the patient population having a DRA of about 4 mm to about 8 mm and an average of about 6 mm. Renal artery vessel length, LRA, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment is important to reach the target neural fibers, the treatment should not be too deep (e.g., >10 mm from inner wall of the artery) to avoid non-target tissue and anatomical structures such as anatomical structures of the digestive system of psoas muscle.

An additional property of the renal artery that may be of interest is the degree of renal motion relative to the aorta induced by respiration and/or blood flow pulsatility. A patient's kidney, which is located at the distal end of the renal artery, may move as much as 10 centimeters cranially with respiratory excursion. This may impart significant motion to the renal artery connecting the aorta and the kidney, thereby requiring from the neuromodulatory apparatus a unique balance of stiffness and flexibility to maintain contact between the energy delivery element and the vessel wall during cycles of respiration. Furthermore, the take-off angle between the renal artery and aorta may vary significantly between patients, and also may vary dynamically within a patient, e.g., due to kidney motion. The take-off angle generally may be in a range of about 30°-135°.

The above detailed descriptions of examples of the technology are not intended to be exhaustive or to limit the technology to the precise form disclosed above. Although specific examples of the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples. All references cited herein are incorporated by reference as if fully set forth herein.

From the foregoing, it will be appreciated that specific examples of the present disclosure have been described herein for purposes of illustration, but that various modifications may be made without deviating from the present disclosure.

Certain aspects of the present disclosure described in the context of particular examples may be combined or eliminated in other examples. Further, while advantages associated with certain examples have been described in the context of those examples, other examples may also exhibit such advantages, and not all examples need necessarily exhibit such advantages to fall within the scope of the present disclosure. Accordingly, the present disclosure and associated technology can encompass other examples not expressly shown or described herein.

Further, although techniques have been described in which a neuromodulation catheter is positioned at a single location within a single renal artery, in other examples, the neuromodulation catheter may be repositioned to a second treatment site within a single renal artery (e.g., proximal or distal of the first treatment site, may be repositioned in a branch of the single artery, may be repositioned within a different renal vessel on the same side of the patient (e.g., a renal vessel associated with the same kidney of the patient), may be repositioned in a renal vessel on the other side of the patient (e.g., a renal vessel associated with the other kidney of the patient), or any combination thereof. At each location where the neuromodulation catheter is positioned, renal neuromodulation may be performed using any of the techniques described herein or any other suitable renal neuromodulation technique or any combination thereof.

Moreover, unless the word “or” is expressly limited to mean only a single term exclusive from the other items in reference to a list of two or more items, then the use of “or” in such a list is to be interpreted as including (a) any single item in list, (b) all of the items in the list, or (c) any combination of the items in the list. Additionally, the terms “about” or approximately,” when preceding a value, should be interpreted to mean plus or minus 10% of the value, unless otherwise indicated. Additionally, the term “comprising” is used throughout to mean including at least the recited feature(s) such that any greater number of the same feature and/or additional types of other features are not precluded. \

The following examples are a non-limiting list of clauses in accordance with one or more techniques of this disclosure.

Example 1. A catheter system comprising: a catheter comprising an elongated body defining a longitudinal axis, the elongated body comprising: an expandable portion; a therapy delivery element disposed on the expandable portion, wherein the expandable portion is configured to transform into an expanded configuration and place the therapy delivery element in apposition with a blood vessel wall at a first location; and a rotation member proximal to and separate from the expandable portion, the rotation member configured to expand to cause the expandable portion to rotate about the longitudinal axis and place the therapy delivery element in apposition with the blood vessel wall at a second location.

Example 2. The catheter system of Example 1, wherein the expandable portion is configured to radially expand away from the longitudinal axis to the expanded configuration.

Example 3. The catheter system of any of Examples 1 and 2, wherein the rotation member is configured to apply a torque to the expandable portion to cause the expandable portion to rotate about the longitudinal axis.

Example 4. The catheter system of any of Examples 1-3, wherein the elongated body comprises a distal portion including the expandable portion, and wherein expansion of the rotation member causes a foreshortening of an axis length of the distal portion along the longitudinal axis.

Example 5. The catheter system of any of Examples 1-4, wherein the therapy delivery element comprises an electrode.

Example 6. The catheter system of any of Examples 1-4, wherein the therapy delivery element comprises an injection port.

Example 7. The catheter system of any of Examples 1-6, wherein the first location and the second location are disposed around an inner perimeter of the blood vessel and are separated by a predetermined angle.

Example 8. The catheter system of Example 7, wherein the predetermined angle is 45 degrees to 180 degrees.

Example 9. The catheter system of Example 8, wherein the predetermined angle is 90 degrees.

Example 10. The catheter system of Example 8, wherein the predetermined angle is 180 degrees.

Example 11. The catheter system of any of Examples 1-10, further comprising a guide member configured to restrain the expandable portion and the rotation member in collapsed configurations.

Example 12. The catheter system of Example 11, wherein the elongated body defines a catheter lumen, and wherein the guide member is configured to be disposed within the catheter lumen.

Example 13. The catheter system of Example 11, wherein the guide member comprises a guide sheath defining a lumen configured to receive the catheter.

Example 14. The catheter system of any of Examples 11-13, wherein the rotation member is configured to expand in response to withdrawal of the guide member to a position proximal to the rotation member.

Example 15. The catheter system of any of Examples 11-14, wherein the expandable portion is configured to expand to the expanded configuration and place the therapy delivery element in apposition with a blood vessel wall at the first location in response to withdrawal of the guide member to a position proximal to the expandable portion and distal to the rotation member.

Example 16. The catheter system of any of Examples 1-15, wherein the expandable portion and the rotation member are self-expandable.

Example 17. The catheter system of any of Examples 1-16, wherein the expanded configuration of the expandable portion comprises a first expanded configuration comprising a first spiral, loop or helix, and wherein the rotation member is configured to expand to a second expanded configuration comprising a second spiral, loop or helix.

Example 18. The catheter system of Example 17, wherein the first spiral, loop or helix and the second spiral, loop or helix are wound in a same direction.

Example 19. The catheter system of Example 17, wherein the first spiral, loop or helix and the second spiral, loop or helix are wound in different directions.

Example 20. The catheter system of any of Examples 17-19, wherein the first expanded configuration defines a larger outer diameter than the second expanded configuration.

Example 21. The catheter system of any of Examples 1-20, wherein the elongated body comprises an elongated tube configured to cause the expandable portion and the rotation member to expand radially away from the longitudinal axis.

Example 22. The catheter system of Example 21, wherein the elongated tube comprises a shape-memory material.

Example 23. The catheter system of any of Examples 1-22, wherein the rotation member comprises a first rotation member, the catheter system further comprising a second rotation member proximal to the first rotation member, wherein the second rotation member is configured to expand to cause the expandable portion to rotate about the longitudinal axis and rotate the therapy delivery element from the second location to a third location.

Example 24. A catheter comprising: an elongated body defining a longitudinal axis, the elongated body comprising: an expandable portion; a therapy delivery element disposed on the expandable portion, wherein the expandable portion is configured to transform from a relatively low-profile configuration to a deployed configuration and place the therapy delivery element in apposition with a blood vessel wall at a first location; and a plurality of rotation members proximal to and separate from the expandable portion, each rotation member of the plurality of rotation members being configured to expand to cause the expandable portion to rotate about the longitudinal axis and place the therapy delivery element in apposition with the blood vessel wall at a corresponding location of a plurality of locations.

Example 25. The catheter of Example 24, wherein the expandable portion is configured to radially expand away from the longitudinal axis to the deployed configuration.

Example 26. The catheter of any of Examples 24 and 25, wherein each rotation member of the plurality of rotation members is configured to apply a torque to the expandable portion to cause the expandable portion to rotate about the longitudinal axis.

Example 27. The catheter of any of Examples 24-26, wherein expansion of each rotation member of the plurality of rotation members causes foreshortening of an axis length of the elongated body along the longitudinal axis.

Example 28. The catheter of any of Examples 24-27, wherein at least two of the plurality of locations are separated by a predetermined angle.

Example 29. The catheter of Example 28, wherein the predetermined angle is 45 degrees to 180 degrees.

Example 30. The catheter of Example 29, wherein the predetermined angle is 180 degrees.

Example 31. The catheter of any of Examples 24-30, where the catheter is configured to be restrained by a guide member disposed over or within at least a portion of the elongated body.

Example 32. The catheter of Example 31, wherein the expandable portion is configured to transform to the deployed configuration in response to a retraction of the guide member to a position proximal to the expandable portion and distal to the plurality of rotation members.

Example 33. The catheter of Example 32, wherein the position is a first position, wherein a first rotation member of the plurality of rotation members is configured to expand in response to retraction of the guide member to a second position proximal to the first rotation member, and wherein a second rotation member of the plurality of rotation members is configured to expand in response to retraction of the guide member to a third position proximal to the second rotation member.

Example 34. The catheter of any of Examples 24-33, wherein the deployed configuration of the expandable portion comprises a first deployed configuration defining a first spiral, loop or helix, and wherein each rotation member of the plurality of rotation members is configured to deploy to a corresponding deployed configuration of a plurality of deployed configurations, each deployed configuration of the plurality of deployed configurations defining a corresponding spiral, loop or helix of a plurality of spirals, loops, or helixes.

Example 35. The catheter of Example 34, wherein the first spiral, loop or helix and one or more spirals, loops, or helixes of the plurality of spirals, loops or helixes are wound in a same direction.

Example 36. The catheter of Example 34, wherein the first spiral, loop or helix and one or more spirals, loops, or helixes of the plurality of spirals, loops or helixes are wound in different directions.

Example 37. The catheter of any of Examples 24-36, wherein the elongated body comprises an elongated tube configured to, when unrestrained, cause one or more of the expandable portion or one or more rotation members of the plurality of rotation members to radially expand away from the longitudinal axis.

Example 38. The catheter of Example 37, wherein the elongated tube comprises a shape-memory material.

Example 39. The catheter of any of Examples 24-38, wherein the expandable portion and the plurality of rotation members are configured to self-expand.

Example 40. The catheter of any of Examples 24-39, wherein the therapy delivery element comprises one or more electrodes.

Example 41. The catheter of any of Examples 24-40, wherein the therapy delivery element comprises one or more injection ports.

Example 2. A method comprising: advancing a catheter through vasculature to a target tissue site within a blood vessel of a patient, the catheter comprising an elongated body defining a longitudinal axis, the elongated body comprising: an expandable portion; a therapy delivery element disposed on the expandable portion; and a rotation member proximal to and separate from the expandable portion; expanding the expandable portion to place the therapy delivery element in apposition to a vessel wall of the blood vessel at a first location; delivering, via the therapy delivery element, a therapy to tissue of the patient through the vessel wall at the first location; expanding the rotation member to rotate the expandable portion within the blood vessel and place the therapy delivery element in apposition to the vessel wall at a second location; and delivering, via the therapy delivery element, the therapy to tissue of the patient through the vessel wall at the second location.

Example 43. The method of Example 42, wherein the first location and the second location are disposed around a circumference of the blood vessel and are separated by a predetermined angle.

Example 44. The method of Example 43, wherein the predetermined angle is 45 degrees to 180 degrees.

Example 45. The method of Example 44, wherein the predetermined angle is 180 degrees.

Example 46. The method of Example 44, wherein the predetermined angle is 90 degrees.

Example 47. The method of any of Examples 42-46, wherein expanding the rotation member comprises applying a torque to the expandable portion to cause the expandable portion to rotate about the longitudinal axis.

Example 48. The method of any of Examples 42-47, wherein the elongated body comprises a distal portion including the expandable portion, and wherein expanding the rotation member foreshortens an axis length of the distal portion of the elongated body along the longitudinal axis.

Example 49. The method of any of Examples 42-48, wherein expanding expandable portion comprises withdrawing a guide member to a first position proximal to the expandable portion and distal to the rotation member to cause expandable portion to expand to the expanded configuration.

Example 50. The method of Example 49, wherein expanding the rotation member comprises withdrawing the guide member to a second position proximal to the rotation member to cause the expandable portion to radially expand away from the longitudinal axis.

Example 51. The method of any of Examples 42-50, wherein expanding the expandable portion comprises expanding the expandable portion to a first expanded configuration comprising a first spiral, loop or helix, and wherein expanding the rotation member comprises expanding the expandable portion to a second expanded configuration comprising a second spiral, loop or helix.

Example 52. The method of any of Examples 42-51, wherein the catheter comprises an elongated tube disposed within the elongated body, the elongated tube comprising a shape-memory material.

Example 53. The method of Example 52, wherein the shape-memory material comprises nitinol.

Example 54. A method of forming the catheter of any of Examples 1-41.

Further disclosed herein is the subject-matter of the following clauses:

1. A catheter system comprising:
a catheter comprising an elongated body defining a longitudinal axis, the elongated body comprising:
   an expandable portion;
   a therapy delivery element disposed on the expandable portion, wherein the expandable portion is configured to transform from a delivery configuration into an expanded configuration and place the therapy delivery element in apposition with a blood vessel wall at a first location; and
   a rotation member proximal to and separate from the expandable portion, the rotation member configured to expand to cause the expandable portion to rotate about the longitudinal axis and place the therapy delivery element in apposition with the blood vessel wall at a second location.

2. The catheter system of clause 1, wherein the expandable portion is configured to radially expand away from the longitudinal axis to the expanded configuration.

3. The catheter system of any of clauses 1 and 2, wherein the rotation member is configured to apply a torque to the expandable portion to cause the expandable portion to rotate about the longitudinal axis.

4. The catheter system of any of clauses 1-3, wherein the elongated body comprises a distal portion including the expandable portion, and wherein expansion of the rotation member causes a foreshortening of an axis length of the distal portion along the longitudinal axis.

5. The catheter system of any of clauses 1-4, wherein the therapy delivery element comprises at least one of an electrode or an injection port.

6. The catheter system of any of clauses 1-5, wherein the first location and the second location are disposed around an inner perimeter of the blood vessel and are separated by a predetermined angle.

7. The catheter system of clause 6, wherein the predetermined angle is between 45 degrees and 180 degrees, or wherein the predetermined angle is 90 degrees, or wherein the predetermined angle is 180 degrees.

8. The catheter system of any of clauses 1-7, further comprising a guide member configured to restrain the expandable portion in the delivery configuration and the rotation member in a collapsed configuration.

9. The catheter system of clause 8, wherein the elongated body defines a catheter lumen, and wherein the guide member is configured to be disposed within the catheter lumen.

10. The catheter system of clause 8, wherein the guide member comprises a guide sheath defining a lumen configured to receive the catheter.

11. The catheter system of any of clauses 8-10, wherein the rotation member is configured to expand in response to withdrawal of the guide member to a position proximal to the rotation member.

12. The catheter system of any of clauses 8-11, wherein the expandable portion is configured to expand to the expanded configuration and place the therapy delivery element in apposition with a blood vessel wall at the first location in response to withdrawal of the guide member to a position proximal to the expandable portion and distal to the rotation member.

13. The catheter system of any of clauses 1-12, wherein the expandable portion and the rotation member are self-expandable.

14. The catheter system of any of clauses 1-24, wherein the expanded configuration of the expandable portion comprises a first expanded configuration comprising a first spiral, loop or helix, and wherein the rotation member is configured to expand to a second expanded configuration comprising a second spiral, loop or helix.

15. The catheter system of clause 14, wherein the first spiral, loop or helix and the second spiral, loop or helix are wound in a same direction, or wherein the first spiral, loop or helix and the second spiral, loop or helix are wound in different directions.

16. The catheter system of any of clauses 14-15, wherein the first expanded configuration defines a larger outer diameter than the second expanded configuration.

17. The catheter system of any of clauses 1-16, wherein the elongated body comprises an elongated tube configured to cause the expandable portion and the rotation member to expand radially away from the longitudinal axis.

18. The catheter system of clause 17, wherein the elongated tube comprises a shape-memory material.

19. The catheter system of any of clauses 1-18, wherein the rotation member comprises a first rotation member, the catheter system further comprising a second rotation member proximal to the first rotation member, wherein the second rotation member is configured to expand to cause the expandable portion to rotate about the longitudinal axis and rotate the therapy delivery element from the second location to a third location.

1. A catheter system comprising:
a catheter comprising an elongated body defining a longitudinal axis, the elongated body comprising:
an expandable portion;
a therapy delivery element disposed on the expandable portion, wherein the expandable portion is configured to transform from a delivery configuration into an expanded configuration and place the therapy delivery element in apposition with a blood vessel wall at a first location; and
a rotation member proximal to and separate from the expandable portion, the rotation member configured to expand to cause the expandable portion to rotate about the longitudinal axis and place the therapy delivery element in apposition with the blood vessel wall at a second location.

2. The catheter system of claim 1, wherein the expandable portion is configured to radially expand away from the longitudinal axis to the expanded configuration.

3. The catheter system of claim 1, wherein the rotation member is configured to apply a torque to the expandable portion to cause the expandable portion to rotate about the longitudinal axis.

4. The catheter system of claim 1, wherein the elongated body comprises a distal portion including the expandable portion, and wherein expansion of the rotation member causes a foreshortening of an axis length of the distal portion along the longitudinal axis.

5. (canceled)

6. The catheter system of claim 1, wherein the first location and the second location are disposed around an inner perimeter of the blood vessel and are separated by a predetermined angle.

7. The catheter system of claim 6, wherein the predetermined angle is between 45 degrees and 180 degrees.

8. The catheter system of claim 1, further comprising a guide member configured to restrain the expandable portion in the delivery configuration and the rotation member in a collapsed configuration.

9-11. (canceled)

12. The catheter system of claim 8, wherein the expandable portion is configured to expand to the expanded configuration and place the therapy delivery element in apposition with the blood vessel wall at the first location in response to withdrawal of the guide member to a position proximal to the expandable portion and distal to the rotation member, and wherein the rotation member is configured to expand and cause the expandable portion to place the therapy delivery element in apposition with the blood vessel wall at the second location in response to withdrawal of the guide member to a position proximal to the rotation member.

13. The catheter system of claim 1, wherein the expandable portion and the rotation member are self-expandable.

14. The catheter system of claim 1, wherein the expanded configuration of the expandable portion comprises a first expanded configuration comprising a first spiral, loop, or helix, and wherein the rotation member is configured to expand to a second expanded configuration comprising a second spiral, loop, or helix.

15. The catheter system of claim 14, wherein the first spiral, loop or helix and the second spiral, loop or helix are wound in a same direction.

16. (canceled)

17. The catheter system of claim 1, wherein the elongated body comprises an elongated tube configured to cause the expandable portion and the rotation member to expand radially away from the longitudinal axis, the elongated tube comprising a shape-memory material.

18. (canceled)

19. The catheter system of claim 1, wherein the rotation member comprises a first rotation member, the catheter system further comprising a second rotation member proximal to the first rotation member, wherein the second rotation member is configured to expand to cause the expandable portion to rotate about the longitudinal axis and rotate the therapy delivery element from the second location to a third location.

20. The catheter system of claim 14, wherein the first spiral, loop, or helix and the second spiral, loop, or helix are wound in different directions.

21. A catheter comprising:
an elongated body defining a longitudinal axis, the elongated body comprising:
an expandable portion;
a therapy delivery element disposed on the expandable portion, wherein the expandable portion is configured to transform from a relatively low-profile configuration to a deployed configuration and place the therapy delivery element in apposition with a blood vessel wall at a first location; and
a plurality of rotation members proximal to and separate from the expandable portion, each rotation member of the plurality of rotation members being configured to expand to cause the expandable portion to rotate about the longitudinal axis and place the therapy delivery element in apposition with the blood vessel wall at a corresponding location of a plurality of locations.

22. The catheter of claim 21, wherein each rotation member of the plurality of rotation members is configured to apply a torque to the expandable portion to cause the expandable portion to rotate about the longitudinal axis.

23. The catheter of claim 21, wherein the deployed configuration of the expandable portion comprises a first deployed configuration defining a first spiral, loop or helix, and wherein each rotation member of the plurality of rotation members is configured to deploy to a corresponding deployed configuration of a plurality of deployed configurations, each deployed configuration of the plurality of deployed configurations defining a corresponding spiral, loop or helix of a plurality of spirals, loops, or helixes.

24. A method comprising:
advancing a catheter through vasculature to a target tissue site within a blood vessel of a patient, the catheter comprising an elongated body defining a longitudinal axis, the elongated body comprising:
an expandable portion;
a therapy delivery element disposed on the expandable portion; and
a rotation member proximal to and separate from the expandable portion;
expanding the expandable portion to place the therapy delivery element in apposition to a vessel wall of the blood vessel at a first location;
delivering, via the therapy delivery element, a therapy to tissue of the patient through the vessel wall at the first location;
expanding the rotation member to rotate the expandable portion within the blood vessel and place the therapy delivery element in apposition to the vessel wall at a second location; and
delivering, via the therapy delivery element, the therapy to tissue of the patient through the vessel wall at the second location.

25. The method of claim 24, wherein expanding the rotation member comprises applying a torque to the expandable portion to cause the expandable portion to rotate about the longitudinal axis.

26. The method of claim 24, wherein expanding the expandable portion comprises expanding the expandable portion to a first expanded configuration comprising a first spiral, loop or helix, and wherein expanding the rotation member comprises expanding the expandable portion to a second expanded configuration comprising a second spiral, loop or helix.
